(19) United States
(12) Patent Application Publication
(10) Pub. No.: US 2022/0339611 A1
(43) Pub. Date: Oct. 27, 2022

(54) Synthesis and Use of a Zeolitic Material Having the ITH Framework Structure Type

(71) Applicant: BASF SE, Ludwigshafen am Rhein (DE)

(72) Inventors: Andrei-Nicolae Parvulescu, Ludwigshafen (DE); Trees Maria De Baerdemaeker, Ludwigshafen (DE); Ulrich Mueller, Ludwigshafen (DE); Feng-Shou Xiao, Hangzhou (CN); Xiangju Meng, Hangzhou (CN); Qinming Wu, Hangzhou (CN)

(21) Appl. No.: 17/761,674

(22) PCT Filed: Sep. 18, 2020
(86) PCT No.: PCT/CN2020/116207

§ 371 Date: Mar. 18, 2022

(30) Foreign Application Priority Data
Sep. 20, 2019 (CN) PCT/CN2019/107018

(51) Int. Cl. B01J 29/70; C01B 33/26; B01J 37/04; B01J 37/00; B01J 37/03; B01J 37/06; B01J 37/02; B01J 37/08; B01J 37/30; B01J 20/18; B01J 20/30; C07C 1/24

(52) U.S. Cl. CPC B01J 29/70; C01B 33/2869; C01B 33/2876; B01J 37/04; B01J 37/0018; B01J 37/031; B01J 37/06; B01J 37/0236; B01J 37/08; B01J 37/30; B01J 20/18; B01J 20/3085; B01J 20/3071; B01J 20/3078; B01J 20/3057; C07C 1/24; C07C 2529/70

(57) ABSTRACT

A zeolitic material having the ITH framework structure type.A process for the preparation of a zeolitic material having the ITH framework structure type, the process comprising: (1) preparing a mixture comprising one or more specific organotemplates as structure direct-ing agents, one or more sources of YO2, optionally one or more sources of X2O3, seed crystals, and a solvent system, wherein Y is tetravalent element and X is a trivalent ele-ment,(2) heating the mixture obtained in (1) for crystallizing a zeolitic material having the ITH framework structure type comprising YO2 and optionally X2O3 in its framework structure; wherein the one or more organotemplates comprise a specific polymeric cation.

### TECHNICAL FIELD

The present invention relates to a process for the preparation of a zeolitic material as well as to a zeolitic material having the ITH framework structure type as such and as obtainable from the inventive process. Furthermore, the present invention relates to the use of the inventive zeolitic materials in specific applications.

### INTRODUCTION

Zeolites have shown important roles in the process of oil refining and fine chemicals production because of their uniform channel distribution, high surface area, and large micropore volume. As an important type of zeolites, germanosilicate-based zeolites provide many new structures, and some structures exhibit excellent performance in various catalytic reactions. In particular, ITH zeolite shows excellent performance in the catalytic cracking and methanol-to-olefins (MTO) reaction.

Because of its unique three dimensional 9×10×10-membered ring pore structure (aperture size of 4.0×4.8, 4.8×5.1, and 4.8×5.3 Å), ITH zeolite has attracted much attention. It could be prepared in the form of silicate and borosilicate but it remained difficult to synthesize the form of aluminosilicate due to competitive growth of EUO zeolite when aluminum exists in the synthesis gel. In order to incorporate aluminum in the structure of ITH zeolite, it was required to add one or more germanium species in the process of ITH zeolite synthesis. However, when a large amount of germanium species exist in the ITH framework structure, the thermal and hydrothermal stability of the respective zeolite is remarkably reduced. In addition, the use of germanium species in the synthesis is costly, which strongly hinders the applications of ITH zeolite as heterogeneous catalysts.

P. Zeng et al. disclose in Microporous and Mesoporous Materials a preparation method of germanium-containing ITQ-13 zeolites, wherein hexamethonium cations were used as structure directing agent.

G. Xu et al. disclose in Microporous and Mesoporous Materials a study on the synthesis of pure silica ITQ-13 zeolite using fumed silica as silica source.

X. Liu et al. disclose in Microporous and Mesoporous Materials a study on the synthesis of allsilica zeolites from highly concentrated gels containing hexamethonium cations. In particular, a synthesis of ITQ-13 is disclosed including fluoride anions in the synthesis gel.

R. Castañeda et al. disclose a preparation method of AI-ITQ-13, wherein hexamethonium cations were used as structure directing agent. ITQ-13 zeolites are disclosed therein being prepared by exchanging boron or germanium with aluminum.

L. Li et al. disclose in Journal of Catalysis AI—Ge-ITQ-13 zeolites with varying contents of Germanium.

H. Ma et al. disclose a study on the reaction mechanism for the conversion of methanol to olefins over H-ITQ-13 zeolite based on density functional theory calculations.

A. Corma et al. disclose in Angewandte Chemie International Edition a study on ITQ-13 zeolites. To solve the problem brought with germanium species, A. Corma et al. also disclosed therein a post-synthesis method by alumination of borosilicate ITH zeolite to form aluminosilicate ITH zeolite.

Q. Wu et al.disclose asolvent-free synthesis of ITQ-13 and other zeolites, wherein hexamethonium dibromide was used as structure directing agent.

CN 106698456 Å, on the other hand, discloses the synthesis of the zeolite Al-ITQ-13 having the ITH type framework structure, wherein a linear polyquarternary ammonium organic template is employed as the structure directing agent. It is disclosed that the molar ratio of H₂O: SiO₂ Al₂O₃: organotemplate: F—in the reaction mixture was in the range of from 1-10:1:0-0.1 0.02-0.06:0.12-0.36.

Thus, an ongoing need remains for an improved synthesis of new zeolitic materials with unique physical and chemical characteristics, in particular in view of their increased use in catalytic applications. Furthermore, there remains still the need for an optimized direct synthesis of a zeolitic material having the ITH framework structure type, in particular for obtaining a zeolitic material being free of germanium, and having a specific silica to alumina ratio in the case where the zeolitic material contains Al in its framework structure. In this regard, the need remains to improve a direct synthesis of a zeolitic material having the ITH framework structure type in view of the used amounts of starting materials, in particular of the organotemplate being a comparatively costly starting material since it usually requires a separate preparation.

### DETAILED DESCRIPTION

It was therefore an object of the present invention to provide a new zeolitic material and a novel method for its synthesis. Furthermore, it was the object of the present invention to provide a new zeolitic material for catalytic applications, in particular for heterogeneous catalysis, and more specifically for the conversion of oxygenates to olefins.

Thus, it has surprisingly been found that a zeolitic material of the ITH framework structure type having specific properties may be directly synthesized using a specific polymeric organotemplate as the structure directing agent. In particular, it has quite unexpectedly been found that using a specific polymeric organotemplate, a zeolitic material of the ITH framework structure type containing a tetravalent element of the zeolitic framework in addition to an optional trivalent element may be directly obtained, whereby the zeolitic material has specific properties including for example a specific molar ratio of the tetravalent element to the trivalent element.

Surprisingly, the zeolitic material having the ITH framework structure type according to the present invention demonstrates excellent hydrothermal stability and good performance in methanol-to-olefin (MTO) reaction. In particular, the zeolitic material of the present invention was characterized in detail, whereby the applied multiple characterization methods (XRD, SEM, TEM, MAS NMR, and NH₃-TPD) show that in particular the zeolitic material comprising Si as tetravalent element and Al as trivalent element (said zeolitic material is also designated as COE-7 or COE-7 zeolite herein) owns very high crystallinity, nanosheet-like crystal morphology, large surface area, fully four-coordinated Al species, and abundant acidic sites. Very interestingly, the COE-7 zeolite of the present invention particularly gives enhanced hydrothermal stability than that of conventional ITH zeolite containing the germanium species. More importantly, catalytic tests in methanol-to-olefin (MTO) reveal that the COE-7 zeolite has much higher selectivity for propylene and longer lifetime than those of commercial ZSM-5 zeolite.

Furthermore, it has surprisingly been found that the zeolitic materials of the present invention display unique properties in catalysis, and in particular in the conversion of oxygenates to olefins, wherein in the conversion of methanol to olefins good C3 selectivities may be achieved.

Therefore, the present invention relates to a zeolitic material having the ITH type framework structure, preferably obtainable and/or obtained according to the process of any one of the embodiments disclosed herein,

wherein the zeolitic material comprises YO₂ and optionally X₂O₃ in its framework structure, wherein Y is a tetravalent element and X is a trivalent element,

wherein the framework structure of the zeolitic material comprises less than 4 weight-% of Ge calculated as GeO₂ and based on 100 weight-% of YO₂ contained in the framework structure, wherein the zeolitic material comprises less than 1.5 weight-% of B calculated as B₂O₃ and based on 100 weight-% of X₂O₃ contained in the framework structure, and

wherein the zeolitic material has a molar ratio YO₂: X₂O₃ of equal or greater than 50.

It is preferred that the zeolitic material comprises YO₂ and X₂O₃ in its framework structure.

In the case where the zeolitic material comprises YO₂ and X₂O₃ in its framework structure, it is preferred that the zeolitic material has a molar ratio YO₂: X₂O₃ of equal or greater than 60, preferably of equal or greater than 100, more preferably in the range of from 100 to 250, more preferably in the range of from 105 to 225, more preferably in the range of from 110 to 200, more preferably in the range of from 120 to 150, more preferably in the range of from 135 to 145.

It is preferred that the framework structure of the zeolitic material comprises less than 3 weight-% of Ge calculated as GeO₂ and based on 100 weight-% of YO₂ contained in the framework structure, preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%.

It is preferred that the zeolitic material comprises less than 3 weight-% of B calculated as B₂03 and based on 100 weight-% of X₂O₃ contained in the framework structure, more preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%.

It is preferred that Y is selected from the group consisting of Si, Sn, Ti, Zr, and mixtures of two or more thereof, Y more preferably being Si and/or Ti, wherein Y is more preferably Si.

It is preferred that X is selected from the group consisting of Al, In, Ga, Fe, and mixtures of two or more thereof, X more preferably being Al and/or Ga, wherein X is more preferably Al.

It is preferred that Y comprises, more preferably consists of, Si.

In the case where Y comprises or consists of Si, it is preferred that the ²⁹Si MAS NMR of the zeolitic material comprises:

a first peak having a maximum in the range of from −101.0 to −107.0 ppm, preferably of from −102.0 to −106.5 ppm, more preferably of from −103.0 to −106.2 ppm, more preferably of from −104.0 to −106.0 ppm, more preferably of from −105.0 to −105.7 ppm, and more preferably of from −105.3 to −105.5 ppm;

a second peak having a maximum in the range of from −105.0 to −112.7 ppm, preferably of from −106.5 to −112.2 ppm, more preferably of from −107.5 to −111.0 ppm, more preferably of from −110.0 to −111.7 ppm, more preferably of from −111.0 to −111.6 ppm, and more preferably of from −111.2 to −111.4 ppm;

a third peak having a maximum in the range of from −111.0 to −116.0 ppm, preferably of from −112.0 to −115.5 ppm, more preferably of from −113.0 to −115.2 ppm, more preferably of from −113.5 to −115.0 ppm, more preferably of from −114.1 to −114.7 ppm, and more preferably of from −114.3 to −114.5 ppm; and

a fourth peak having a maximum in the range of from −115.1 to −118.4 ppm, preferably of from −115.6 to −117.9 ppm, more preferably of from −116.1 to −117.4 ppm, more preferably of from −116.4 to −117.1 ppm, and more preferably of from −116.6 to −116.9 ppm, wherein preferably the ²⁹Si MAS NMR of the zeolitic material comprises only four peaks in the range of from −80 to −130 ppm. The ²⁹Si MAS NMR of the zeolitic material is preferably determined according to reference example 6 disclosed herein.

It is preferred that the zeolitic material comprises F.

In the case where the zeolitic material comprises F, it is preferred that the ¹⁹F MAS NMR of the zeolitic material comprises:

a first peak having a maximum in the range of from −32 to −38 ppm, preferably in the range of from −33.0 to −37.4 ppm, more preferably in the range of from −34.0 to −36.0 ppm, more preferably in the range of from −35.0 to −36.0 ppm, a second peak having a maximum in the range of from −61.3 to −66.3 ppm, preferably in the range of from −61.0 to −65.8 ppm, more preferably of from −62.3 to −65.3 ppm, more preferably of from −62.8 to −64.8 ppm, more preferably of from −63.3 to −64.3 ppm;

wherein preferably the ¹⁹F MAS NMR of the zeolitic material comprises only two peaks in the range of from 0 to −100 ppm. The ¹⁹F MAS NMR of the zeolitic material is preferably determined according to reference example 6 disclosed herein.

It is preferred that X comprises, more preferably consists of, Al.

In the case where X comprises or consists of Al, it is preferred that the ²⁷AI MAS NMR of the zeolitic material comprises:

a peak having a maximum in the range of from 50 to 58 ppm, preferably of from 51 to 57 ppm, more preferably of from 52 to 56 ppm, more preferably of from 52.5 to 55.5 ppm, more preferably of from 53 to 55 ppm,

wherein preferably the ²⁷AI MAS NMR of the zeolitic material comprises a single peak having a maximum in the range of from −40 to 140 ppm. The ²⁷AI MAS NMR of the zeolitic material is preferably determined according to reference example 6 disclosed herein.

According to a first alternative, it is preferred that the zeolitic material, preferably the calcined zeolitic material, displays an X-ray diffraction pattern comprising at least the following reflections:

| | Diffraction angle 2θ/° |
|---|---|
| Intensity (%) | [Cu K(alpha 1)] |
| 100 | [7.00-7.20] |
| [33-53] | [7.82-8.02] |
| [22-42] | [8.60-8.80] |
| [42-62] | [11.12-11.32] |
| [28-48] | [15.38-15.58] |
| [19-39] | [16.05-16.25] |
| [19-39] | [16.48-16.68] |
| [17-37] | [20.94-21.14] |
| [14-34] | [21.40-21.60] |
| [77-97] | [22.92-23.12] |
| [57-77] | [23.54-23.74] |
| [31-51] | [23.90-24.10] |

wherein 100% relates to the intensity of the maximum peak in the X-ray powder diffraction pattern, and wherein the X-ray diffraction pattern is preferably determined according to reference example 2 disclosed herein.

It is particularly preferred that the zeolitic material, preferably the calcined zeolitic material, displays an X-ray diffraction pattern comprising at least the following reflections:

| | Diffraction angle 2θ/° |
|---|---|
| Intensity (%) | [Cu K(alpha 1)] |
| 100 | [7.05-7.15] |
| [33-53] | [7.87-7.97] |
| [22-42] | [8.65-8.75] |
| [42-62] | [11.17-11.27] |
| [28-48] | [15.43-15.53] |
| [19-39] | [16.10-16.20] |
| [19-39] | [16.53-16.63] |
| [17-37] | [20.99-21.09] |
| [14-34] | [21.45-21.55] |
| [77-97] | [22.97-23.07] |
| [57-77] | [23.59-23.69] |
| [31-51] | [23.95-24.05] |

wherein 100% relates to the intensity of the maximum peak in the X-ray powder diffraction pattern, and wherein the X-ray diffraction pattern is preferably determined according to reference example 2 disclosed herein.

According to a second alternative, it is preferred that the zeolitic material, preferably the calcined zeolitic material, displays an X-ray powder diffraction pattern comprising at least the following reflections:

| | Diffraction angle 2θ/° |
|---|---|
| Intensity (%) | [Cu K(alpha 1)] |
| [24-44] | [8.09-8.29] |
| [25-45] | [11.29-11.49] |
| [20-40] | [16.08-16.28] |
| [33-53] | [21.13-21.33] |
| [28-48] | [21.51-21.71] |
| [24-44] | [22.45-22.65] |
| 100 | [23.10-23.30] |
| [67-87] | [23.77-23.97] |
| [44-64] | [24.19-24.39] |
| [18-38] | [27.76-27.96] |

wherein 100% relates to the intensity of the maximum peak in the X-ray powder diffraction pattern, and wherein the X-ray powder diffraction pattern is preferably determined according to reference example 2 disclosed herein, wherein preferably the zeolitic material, more preferably the calcined zeolitic material, displays an X-ray powder diffraction pattern comprising at least the following reflections:

| | Diffraction angle 2θ/° |
|---|---|
| Intensity (%) | [Cu K(alpha 1)] |
| [29-39] | [8.14-8.24] |
| [30-40] | [11.34-11.44] |
| [25-35] | [16.13-16.23] |
| [38-48] | [21.18-21.28] |
| [33-43] | [21.56-21.66] |
| [29-39] | [22.50-22.60] |
| 100 | [23.15-23.25] |
| [72-82] | [23.82-23.92] |
| [49-59] | [24.24-24.34] |
| [23-33] | [27.81-27.91] |

wherein 100% relates to the intensity of the maximum peak in the X-ray powder diffraction pattern, and wherein the X-ray powder diffraction pattern is preferably determined according to reference example 2 disclosed herein.

It is preferred that the BET surface area of the zeolitic material is in the range of from 50 to 800 m²/g, more preferably from 100 to 700 m²/g, more preferably from 200 to 600 m²/g, more preferably from 300 to 500 m²/g, more preferably from 350 to 450 m²/g, more preferably from 375 to 425 m²/g, more preferably from 390 to 410 m²/g, more preferably from 395 to 405 m²/g, wherein preferably the BET surface area is determined according to ISO 9277:2010.

It is preferred that the micropore volume of the zeolitic material is in the range of from 0.05 to 0.5 cm³/g, more preferably from 0.075 to 0.3 cm³/g, more preferably from 0.1 to 0.25 cm³/g, more preferably from 0.11 to 0.19 cm³/g, more preferably from 0.13 to 0.17 cm³/g, and more preferably from 0.14 to 0.16 cm³/g, wherein preferably the micropore volume is determined according to ISO 15901-1:2016.

It is preferred that the mesopore volume of the zeolitic material is in the range of from 0.05 to 0.5 cm³/g, more preferably from 0.1 to 0.3 cm³/g, more preferably from 0.18 to 0.26 cm³/g, more preferably from 0.20 to 0.24 cm³/g, and more preferably from 0.21 to 0.23 cm³/g, wherein preferably the mesopore volume is determined according to ISO 15901-3:2007.

It is preferred that the zeolitic material has a nanosheet-like crystal morphology. In the case where the zeolitic material has a nanosheet-like crystal morphology, it is preferred that the thickness of a nanosheet is in the range of from 5 to 100 nm, more preferably in the range of from 10 to 50 nm, more preferably in the range of from 25 to 35 nm, preferably determined according to reference example 4 and/or according to reference example 5 disclosed herein.

It is preferred that the zeolitic material shows in the temperature programmed desorption of ammonia (NH₃-TPD)

a first desorption peak centered in the range of from 170 to 200° C., preferably in the range of from 180 to 190° C., more preferably in the range of from 183 to 187° C., and

a second desorption peak centered in the range of from 370 to 410° C., preferably in the range of from 380 to 400° C., more preferably in the range of from 385 to 395° C., preferably determined according to reference example 7 disclosed herein.

It is preferred that the zeolitic material comprises one or more metal cations M at the ionexchange sites of the framework structure of the zeolitic material, wherein the one or more metal cations M are more preferably selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, preferably selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, more preferably from the group consisting of Sr, Cr, Mg, Ca, Mo, Fe, Co, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Cr, Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, and more preferably from the group consisting of Fe, Cu, Mg, Ca, Zn, Mo, La, Ce, Pr, Nd, Sm, Eu, Gd, Tb, Dy, Ho, Er, Tm, Yb, Lu, Y, Sc, and mixtures of two or more thereof.

In the case where the zeolitic material comprises one or more metal cations M at the ionexchange sites of the framework structure of the zeolitic material, it is preferred that the zeolitic material comprises the one or more metal cations M in an amount in the range of from 0.01 to 10 weight-% based on 100 weight-% of Si in the zeolitic material calculated as SiO₂, more preferably in the range of from 0.05 to 7 weight-%, more preferably in the range of from 0.1 to 5 weight-%, more preferably in the range of from 0.5 to 4.5 weight-%, more preferably in the range of from 1 to 4 weight-%, more preferably in the range of from 1.5 to 3.5 weight-%.

It is preferred that from 95 to 100 weight-% of the zeolitic material consists of Si, optionally Al, 0, H, and the one or more metal cations M, calculated based on the total weight of the zeolitic material, more preferably from 97 to 100 weight-%, more preferably from 99 to 100 weight-%.

It is preferred that from 95 to 100 weight-% of the framework of the zeolitic material consists of Si, optionally Al, 0, and H, based on the total weight of the framework of the zeolitic material, more preferably from 97 to 100 weight-%, more preferably from 99 to 100 weight-%.

Further, the present invention relates to a process for the preparation of a zeolitic material having the ITH framework structure type, preferably of a zeolitic material according to any one of the embodiments disclosed herein, wherein the process comprises

(1) preparing a mixture comprising one or more organotemplates as structure directing agents, one or more sources of YO₂, optionally one or more sources of X₂03, seed crystals, and a solvent system, wherein Y is tetravalent element and X is a trivalent element;

(2) heating the mixture obtained in (1) for crystallizing a zeolitic material having the ITH framework structure type comprising YO₂ and optionally X₂O₃ in its framework structure; wherein the one or more organotemplates comprise a polymeric cation comprising a unit of formula (I):

[R¹R²N+−⁵−N+R³R⁴−R⁶], (I);

wherein R¹, R², R³, and R⁴ independently from one another is (C₁-C₄)alkyl, preferably (C₁-C₃)alkyl, more preferably ethyl or methyl, and more preferably methyl;

wherein R⁵ is selected from the group consisting of tetramethylene, pentamethylene, hexamethylene, and heptamethylene, wherein preferably R⁵ is pentamethylene or hexamethylene, wherein more preferably R⁵ is hexamethylene;

wherein R⁶ is selected from the group consisting of trimethylene, tetramethylene, and pentamethylene, wherein preferably R⁶ is trimethylene or tetramethylene, wherein more preferably R⁶ is tetramethylene;

wherein n is a natural number in the range of from 1 to 50, preferably in the range of from 2 to 40, more preferably in the range of from 5 to 30, more preferably in the range of from 10 to 23, more preferably in the range of from 11 to 22.

It is preferred that the organotemplate: YO₂ molar ratio of the one or more organotemplates to the one or more sources of YO₂ calculated as YO₂ in the mixture prepared in (1) and heated in (2) is in the range of from 0.001 to 0.5, more preferably from 0.0012 to 0.27, more preferably from 0.0015 to 0.24, more preferably from 0.002 to 0.2, more preferably from 0.0025 to 0.1, more preferably from 0.003 to 0.02, more preferably from 0.0035 to 0.015, more preferably from 0.004 to 0.01, and more preferably from 0.0045 to 0.006.

It is preferred that the one or more organotemplates are provided as salts, more preferably as one or more salts selected from the group consisting of halides, sulfate, nitrate, phosphate, acetate, hydroxide, and mixtures of two or more thereof, more preferably from the group consisting of bromide, chloride, hydroxide, sulfate, and mixtures of two or more thereof, wherein more preferably the one or more organotemplates are provided as hydroxides and/or bromides, and more preferably as hydroxides.

It is preferred that Y is selected from the group consisting of Si, Sn, Ti, Zr, Ge, and mixtures of two or more thereof, Y more preferably being Si and/or Ti, wherein Y is more preferably Si.

It is preferred that X is selected from the group consisting of Al, B, In, Ga, and mixtures of two or more thereof, more preferably from the group consisting of Al, B, Ga, and mixtures of two or more thereof, X more preferably being Al and/or B, wherein X is more preferably Al.

It is preferred that the seed crystals comprise one or more zeolitic materials having the ITH framework structure type, wherein more preferably the seed crystals comprise ITQ-13, wherein more preferably the seed crystals consist of one or more zeolitic materials having the ITH framework structure type, wherein more preferably the seed crystals consist of ITQ-13.

It is preferred that the seed crystals comprise one or more zeolitic materials having the ITH framework structure type, more preferably one or more zeolitic materials according to any one of the embodiments disclosed herein, wherein more preferably the seed crystals consist of one or more zeolitic materials having the ITH framework structure type, wherein more preferably the seed crystals consist of one or more zeolitic materials according to any one of the embodiments disclosed herein.

It is preferred that the seed crystals comprise one or more zeolitic materials having the ITH framework structure type, more preferably one or more zeolitic materials having the ITH framework structure type, wherein from 95 to 100 weight-% of the one or more zeolitic materials having the ITH framework structure type consist of Si, O, and H, more preferably from 97 to 100 weight-%, more preferably from 99 to 100 weight-%.

It is preferred that the amount of seed crystals comprised in the mixture prepared in (1) is in the range of from 0.1 to 15 weight-% based on 100 weight-% of the one or more sources of YO₂ calculated as YO₂, more preferably from 0.5 to 12 weight-%, more preferably from 1 to 10 weight-%, more preferably from 2 to 8 weight-%, more preferably from 3 to 7 weight-%, more preferably from 5 to 6 weight-%.

It is preferred that the mixture prepared in (1) and heated in (2) contains less than 5 weight-% of Ge calculated as GeO₂ and based on 100 weight-% of the one or more sources of YO₂ calculated as YO₂, more preferably less than 3 weight-%, more preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%.

It is preferred that the mixture prepared in (1) and heated in (2) contains less than 5 weight-% of B calculated as B₂O₃ and based on 100 weight-% of the one or more sources of X₂O₃ calculated as X₂03, more preferably less than 3 weight-%, more preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%.

It is preferred that the mixture comprises one or more sources for X₂03, wherein the X₂O₃: YO₂ molar ratio of the one or more sources of X₂O₃ calculated as X₂O₃ to the one or more sources of YO₂ calculated as YO₂ in the mixture prepared in (1) and heated in (2) is in the range of from 0.001 to 0.1, more preferably of from 0.0015 to 0.05, more preferably of from 0.0017 to 0.030, more preferably of from 0.0019 to 0.015, more preferably of from 0.002 to 0.01, more preferably of from 0.0025 to 0.007.

It is preferred that the mixture prepared in (1) further comprises one or more sources of fluoride, wherein more preferably the fluoride: YO₂ molar ratio of the one or more sources of fluoride calculated as the element to the one or more sources of YO₂ calculated as YO₂ in the mixture prepared in (1) and heated in (2) is in the range of from 0.01 to 2, preferably from 0.05 to 1.5, more preferably from 0.1 to 1, more preferably from 0.13 to 0.55, more preferably from 0.14 to 0.45, more preferably from 0.15 to 0.4, more preferably from 0.2 to 0.3.

In the case where the mixture prepared in (1) further comprises one or more sources of fluoride, it is preferred that the one or more sources of fluoride is selected from fluoride salts, HF, and mixtures of two or more thereof, more preferably from the group consisting of alkali metal fluoride salts, ammonium fluoride salts, HF, and mixtures of two or more thereof, wherein more preferably the one or more sources of fluoride comprise HF or ammonium fluoride, wherein more preferably the one or more sources of fluoride comprise HF, wherein more preferably the one or more sources of fluoride consist of HF.

It is preferred that the one or more sources for YO₂ comprises one or more compounds selected from the group consisting of fumed silica, silica hydrosols, reactive amorphous solid silicas, silica gel, silicic acid, water glass, sodium metasilicate hydrate, sesquisilicate, disilicate, colloidal silica, silicic acid esters, and mixtures of two or more thereof, more preferably from the group consisting of fumed silica, silica hydrosols, silica gel, silicic acid, water glass, sodium metasilicate hydrate, sesquisilicate, disilicate, colloidal silica, tetra(C₁-C₄)alkylorthosilicate, and mixtures of two or more thereof, more preferably from the group consisting of fumed silica, silica hydrosols, silicic acid, tetra(C₂-C₃)alkylorthosilicate, and mixtures of two or more thereof, wherein more preferably the one or more sources for YO₂ fumed silica, wherein more preferably the one or more sources for YO₂ consist of fumed silica.

According to a first alternative, it is preferred that the one or more sources for X₂O₃ comprises one or more compounds selected from the group consisting of alumina, aluminates, aluminum salts, and mixtures of two or more thereof, more preferably from the group consisting of alumina, aluminum salts, and mixtures of two or more thereof, more preferably from the group consisting of alumina, aluminum tri(C₁-C₅)alkoxide, AIO(OH), AI(OH)₃, aluminum halides, preferably aluminum fluoride and/or chloride and/or bromide, more preferably aluminum fluoride and/or chloride, and even more preferably aluminum chloride, aluminum sulfate, aluminum phosphate, aluminum fluorosilicate, and mixtures of two or more thereof, more preferably from the group consisting of aluminum tri(C₂-C₄)alkoxide, AIO(OH), AI(OH)₃, aluminum chloride, aluminum sulfate, aluminum phosphate, and mixtures of two or more thereof, more preferably from the group consisting of aluminum tri(C₂-C₃)alkoxide, AIO(OH), AI(OH)₃, aluminum chloride, aluminum sulfate, and mixtures of two or more thereof, more preferably from the group consisting of aluminum tripropoxides, AIO(OH), aluminum sulfate, and mixtures of two or more thereof, wherein more preferably the one or more sources for X₂O₃ comprises AIO(OH), and wherein more preferably the one or more sources for X₂O₃ consist of AIO(OH), preferably gamma-AIO(OH).

According to a second alternative, it is preferred that the one or more sources for X₂O₃ comprises a zeolitic material comprising YO₂ and X₂O₃ in its framework structure, wherein Y is tetravalent element and X is a trivalent element; wherein Y is preferably selected from the group consisting of Si, Sn, Ti, Zr, Ge, and mixtures of two or more thereof, Y more preferably being Si and/or Ti, more preferably Si; wherein X is preferably selected from the group consisting of Al, B, In, Ga, and mixtures of two or more thereof, more preferably from the group consisting of Al, B, Ga, and mixtures of two or more thereof, X more preferably being Al and/or B, more preferably Al; wherein the zeolitic material has a molar ratio YO₂: X₂O₃ of equal or greater than 0.1, preferably in the range of from 0.3 to 100, more preferably in the range of from 0.5 to 50, more preferably in the range of from 0.7 to 10, more preferably in the range of from 0.9 to 5, more preferably in the range of from 1 to 3; wherein the zeolitic material preferably has a framework structure type selected from the group consisting of FAU, GIS, MOR, LTA, FER, TON, MTT, BEA, MEL, MWW, MFS, MFl, mixed structures of two or more thereof, and a mixture of two or more thereof, more preferably selected from the group consisting of FAU, GIS, MOR, LTA, FER, TON, MTT, BEA, MEL, MWW, MFS, MFl, mixed structures of two or more thereof, and a mixture of two or more thereof, more preferably an FAU and/or a LTA framework structure type.

In the case where the one or more sources for X₂O₃ comprises a zeolitic material comprising YO₂ and X₂O₃ in its framework structure, it is preferred that the zeolitic material having an LTAtype framework structure type is selected from the group consisting of Linde Type A (zeolite A), Alpha, [AI—Ge-O]-LTA, N-A, LZ-215, SAPO-42, ZK-4, ZK-21, Dehyd. Linde Type A (dehyd. zeolite A), ZK-22, ITQ-29, UZM-9, including mixtures of two or more thereof, preferably from the group consisting of Linde Type A, Alpha, N-A, LZ-215, SAPO-42, ZK-4, ZK-21, Dehyd. Linde Type A, ZK-22, ITQ-29, UZM-9, including mixtures of two or more thereof, more preferably from the group consisting of Linde Type A, Alpha, N-A, LZ-215, ZK-4, ZK-21, Dehyd. Linde Type A, ZK-22, ITQ-29, UZM-9, including mixtures of two or more thereof, more preferably from the group consisting of Linde Type A, Alpha, N-A, LZ-215, ZK-4, ZK-21, ZK-22, ITQ-29, UZM-9, including mixtures of two or more thereof.

Further in the case where the one or more sources for X₂O₃ comprises a zeolitic material comprising YO₂ and X₂O₃ in its framework structure, it is preferred that the zeolitic material having an FAU framework structure type is selected from the group consisting of ZSM-3, Faujasite, [Al—Ge-O]-FAU, CSZ-1, ECR-30, Zeolite X, Zeolite Y, LZ-210, SAPO-37, ZSM-20, Na—X, US-Y, NaY, [Ga—Ge-O]-FAU, Li-LSX, [Ga-AI—Si-O]-FAU, [Ga—Si-O]-FAU, and a mixture of two or more thereof, preferably from the group consisting of ZSM-3, Faujasite, CSZ-1, ECR-30, Zeolite X, Zeolite Y, LZ-210, ZSM-20, Na—X, US-Y, Na—Y, Li-LSX, and a mixture of two or more thereof, more preferably from the group consisting of Faujasite, Zeolite X, Zeolite Y, Na—X, US-Y, Na—Y, and a mixture of two or more thereof, more preferably from the group consisting of Faujasite, Zeolite X, Zeolite Y, and a mixture of two or more thereof, wherein more preferably the zeolitic material having an FAU framework structure type comprises Zeolite X and/or Zeolite Y, preferably Zeolite X, wherein more preferably the zeolitic material having an FAU framework structure type is Zeolite X and/or Zeolite Y, preferably Zeolite X.

It is preferred that the solvent system is selected from the group consisting of optionally branched (C₁-C₄)alcohols, distilled water, and mixtures thereof, more preferably from the group consisting of optionally branched (C₁-C₃)alcohols, distilled water, and mixtures thereof, more preferably from the group consisting of methanol, ethanol, distilled water, and mixtures thereof, wherein more preferably the solvent system comprises distilled water, wherein more preferably the solvent system consists of distilled water.

It is preferred that the H₂O: YO₂ molar ratio of H₂O to the one or more sources of YO₂ calculated as YO₂ in the mixture prepared in (1) and heated in (2) is in the range of from 0.1 to 15, more preferably from 0.2 to 7.5, more preferably from 0.4 to 5, more preferably from 0.5 to 4, more preferably from 0.9 to 3.1, more preferably from 1 to 3.

It is preferred that heating in (2) is conducted for a duration in the range of from 10 min to 35 d, more preferably of from 1 h to 30 d, more preferably from 2 d to 25 d, more preferably from 5 d to 20 d, more preferably from 6 d to 15 d, more preferably from 7 d to 13 d, more preferably from 9 d to 11 d, and more preferably from 9.5 to 10.5 d.

It is preferred that heating in (2) is conducted at a temperature in the range of from 80 to 220° C., more preferably of from 110 to 200° C., more preferably of from 130 to 190° C., more preferably of from 140 to 180° C., more preferably from 145 to 175° C., more preferably of from 150 to 170° C., and more preferably of from 155 to 165° C.

It is preferred that heating in (2) is conducted under autogenous pressure, more preferably under solvothermal conditions, more preferably under hydrothermal conditions, wherein preferably heating in (2) is performed in a pressure tight vessel, preferably in an autoclave.

It is preferred that the one or more organotemplates are prepared according to a process comprising

(a) preparing a reaction mixture comprising a compound having the formula (II)

R¹R²N+−R⁵−N+R³R⁴ (II)

a compound having the formula (III)

Rₐ−R⁶−R_{b} (III)

and a solvent system, to obtain a reaction mixture;

(b) heating the reaction mixture, to obtain a mixture comprising one or more organotemplates; wherein R¹, R², R³, and R⁴ independently from one another is (C₁-C₄)alkyl, preferably (C₁-C₃)alkyl, more preferably ethyl or methyl, and more preferably methyl;

wherein R⁵ is selected from the group consisting of tetramethylene, pentamethylene, hexamethylene, and heptamethylene, wherein preferably R⁵ is pentamethylene or hexamethylene, wherein more preferably R⁵ is hexamethylene;

wherein R⁶ is selected from the group consisting of trimethylene, tetramethylene, and pentamethylene, wherein preferably R⁶ is trimethylene or tetramethylene, wherein more preferably R⁶ is tetramethylene; and

wherein Rₐ and R_{b} independently from each other is selected from the group consisting of F, C1, Br, I, tosyl (OTs), mesyl, triflourmethansulfonate (OTf), and OH, preferably from the group consisting of F, C1, Br, I, and OH, more preferably from the group consisting of Br, I, and OH, more preferably Ra and R_{b} independently from each other is Br.

In the case where the one or more organotemplates are prepared according to a process as disclosed herein, it is preferred that a molar ratio of the compound having the formula (II) to the compound having the formula (III) in the mixture in (a) is in the range of from 0.1:1 to 10:1, more preferably in the range of from 0.5:1 to 2:1, more preferably in the range of from 0.9:1 to 1.1:1.

Further in the case where the one or more organotemplates are prepared according to a process as disclosed herein, it is preferred that heating in (b) is conducted of reflux of the solvent system, wherein more preferably heating in (b) is conducted at a temperature in the range of from 50 to 110° C., preferably in the range of from 70 to 90° C., more preferably in the range of from 75 to 85° C.

Further in the case where the one or more organotemplates are prepared according to a process as disclosed herein, it is preferred that heating in (b) is conducted for a duration in the range of from 1 to 25 h, more preferably from 9 to 15 h, more preferably from 11 to 13 h. Further in the case where the one or more organotemplates are prepared according to a process as disclosed herein, it is preferred that the solvent system comprises one or more of water, methanol, ethanol, propanol, and tetrahydrofuran, more preferably one or more of methanol, ethanol, and propanol, more preferably ethanol, wherein more preferably the solvent system consists of ethanol.

Further in the case where the one or more organotemplates are prepared according to a process as disclosed herein, it is preferred that the process further comprises

(c) isolating one or more organotemplates from the mixture obtained in (b), and/or

(d) washing the one or more organotemplates obtained in (b) or (c).

In the case where the one or more organotemplates are prepared according to a process comprising (c), it is preferred that isolating in (c) is conducted by filtration.

In the case where the one or more organotemplates are prepared according to a process comprising (d), it is preferred that washing in (d) is conducted with one or more of diethylether, tetrahydrofuran, and ethyl acetate, more preferably with diethyl ether.

It is preferred that the process further comprises

(3) isolating the zeolitic material obtained in (2), and/or

(4) washing the zeolitic material obtained in (2) or (3), and/or

(5) drying the zeolitic material obtained in (2), (3), or (4), in a gas atmosphere, and/or

(6) calcining the zeolitic material obtained in (2), (3), (4) or (5) in a gas atmosphere, and/or

(7) subjecting the zeolitic material obtained in (2), (3), (4), (5) or (6) to an ion-exchange procedure with one or more metal cations M, wherein the steps (3) and/or (4) and/or (5) and/or (6) and/or (7) can be conducted in any order, and wherein one or more of said steps is preferably repeated one or more times.

In the case where the process comprises (7), it is preferred that the one or more metal cations M are selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, more preferably selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, more preferably from the group consisting of Sr, Cr, Mg, Ca, Mo, Fe, Co, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Cr, Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, and more preferably from the group consisting of Fe, Cu, Mg, Ca, Zn, Mo, La, Ce, Pr, Nd, Sm, Eu, Gd, Tb, Dy, Ho, Er, Tm, Yb, Lu, Y, Sc, and mixtures of two or more thereof, wherein the one or more metal cations M are located at the ionexchange sites of the framework structure of the zeolitic material.

In the case where the process further comprises (5), it is preferred that drying in (5) is conducted at a temperature of the gas atmosphere in the range of from 60 to 140° C., preferably of from 80 to 120° C., and more preferably of from 90 to 110° C.

Further in the case where the process further comprises (5), it is preferred that the gas atmosphere for drying in (5) comprises nitrogen, oxygen, or a mixture thereof, wherein the gas atmosphere is preferably oxygen, air, or lean air.

Further in the case where the process comprises (6), it is preferred that calcination in (6) is conducted for a duration in the range of from 0.5 to 15 h, more preferably of from 1 to 10 h, more preferably of from 2 to 8 h, more preferably of from 3 to 7 h, more preferably of from 3.5 to 6.5 h, more preferably of from 4 to 6 h, more preferably of from 4.5 to 5.5 h.

Further in the case where the process comprises (6), it is preferred that the gas atmosphere for calcination in (6) comprises nitrogen, oxygen, or a mixture thereof, wherein the gas atmosphere is more preferably oxygen, air, or lean air.

Further in the case where the process comprises (6), it is preferred that calcination in (6) is conducted at a temperature of the gas atmosphere in the range of from 300 to 800° C., more preferably of from 375 to 725° C., more preferably of from 425 to 675° C., more preferably of from 475 to 625° C., and more preferably of from 525 to 575° C.

Yet further, the present invention relates to a zeolitic material having the ITH framework structure type obtainable and/or obtained from the process of any one of the embodiments disclosed herein.

Yet further, the present invention relates to a method for the conversion of oxygenates to olefins comprising

(i) providing a catalyst according to any one of the embodiments disclosed herein;

(ii) providing a gas stream comprising one or more oxygenates and optionally one or more olefins and/or optionally one or more hydrocarbons;

(iii) contacting the catalyst provided in (i) with the gas stream provided in (ii) and converting one or more oxygenates to one or more olefins and optionally to one or more hydrocarbons;

(iv) optionally recycling one or more of the one or more olefins and/or of the one or more hydrocarbons contained in the gas stream obtained in (iii) to (ii).

It is preferred that the catalyst is provided in a fixed bed or in a fluidized bed.

It is preferred that the gas stream provided in (ii) comprises one or more oxygenates selected from the group consisting of aliphatic alcohols, ethers, carbonyl compounds and mixtures of two or more thereof, more preferably from the group consisting of (C1-C₆) alcohols, di(C₁-C₃)alkyl ethers, (C1-C₆) aldehydes, (C₂-C₆) ketones and mixtures of two or more thereof, more preferably consisting of (C1-C₄) alcohols, di(C₁-C₂)alkyl ethers, (C1-C₄) aldehydes, (C₂-C₄) ketones and mixtures of two or more thereof, more preferably from the group consisting of methanol, ethanol, n-propanol, isopropanol, butanol, dimethyl ether, diethyl ether, ethyl methyl ether, diisopropyl ether, di-n-propyl ether, formaldehyde, dimethyl ketone and mixtures of two or more thereof, more preferably from the group consisting of methanol, ethanol, dimethyl ether, diethyl ether, ethyl methyl ether and mixtures of two or more thereof, the gas stream more preferably comprising methanol and/or dimethyl ether, more preferably methanol.

It is preferred that the content of oxygenates in the gas stream provided in (ii) is in the range of from 2 to 100% by volume based on the total volume, more preferably from 3 to 99% by volume, more preferably from 4 to 95% by volume, more preferably from 5 to 80% by volume, more preferably from 6 to 50% by volume.

It is preferred that the gas stream provided in (ii) comprises water, wherein the water content in the gas stream provided in (ii) is more preferably in the range from 5 to 60% by volume, more preferably from 10 to 50% by volume.

It is preferred that the gas stream provided in (ii) further comprises one or more diluting gases, more preferably one or more diluting gases in an amount ranging from 0.1 to 90% by volume, more preferably from 1 to 85% by volume, more preferably from 5 to 80% by volume, more preferably from 10 to 75% by volume.

It is preferred that the one or more diluting gases are selected from the group consisting of H₂O, helium, neon, argon, krypton, nitrogen, carbon monoxide, carbon dioxide, and mixtures of two or more thereof, more preferably from the group consisting of H₂O, argon, nitrogen, carbon dioxide, and mixtures of two or more thereof, wherein more preferably the one or more diluting gases comprise H₂O, wherein more preferably the one or more diluting gases is H₂O.

It is preferred that contacting according to (iii) is effected at a temperature in the range from 225 to 700° C., more preferably from 275 to 650° C., more preferably from 325 to 600° C., more preferably from 375 to 550° C., more preferably from 425 to 525° C., and more preferably from 450 to 500° C.

It is preferred that contacting according to (iii) is effected at a pressure in the range from 0.01 to 25 bar, more referably from 0.1 to 20 bar, more preferably from 0.25 to 15 bar, more preferably from 0.5 to 10 bar, more preferably from 0.75 to 5 bar, more preferably from 0.8 to 2 bar, more preferably from 0.85 to 1.5 bar, more preferably from 0.9 to 1.1 bar.

It is preferred that the method is a continuous method. In the case where the method is a continuous method, it is preferred that the gas hourly space velocity (GHSV) in the contacting in (iii) is preferably in the range from 1 to 30,000 h-¹, more preferably from 500 to 25,000 h-¹, preferably from 1,000 to 20,000 h-¹, more preferably from 1,500 to 10,000 h-¹, more preferably from 2,000 to 5,000 h-¹.

It is preferred that the one or more olefins and/or one or more hydrocarbons optionally provided in (ii) and/or optionally recycled to (ii) comprise one or more selected from the group consisting of ethylene, (C₄—C,)olefins, (C₄—C,)hydrocarbons, and mixtures of two or more thereof, and more preferably from the group consisting of ethylene, (C₄-C₅)olefins, (C₄-C₅)hydrocarbons, and mixtures of two or more thereof.

Yet further the present invention relates to a use of a zeolitic material according to any one of the embodiments disclosed herein as a molecular sieve, as an adsorbent, for ion-exchange, or as a catalyst and/or as a catalyst support, more preferably as a catalyst for the selective catalytic reduction (SCR) of nitrogen oxides NOx; for the oxidation of NH₃, in particular for the oxidation of N H₃ slip in diesel systems; for the decomposition of N₂0; as an additive in fluid catalytic cracking (FCC) processes; and/or as a catalyst in organic conversion reactions, preferably as a hydrocracking catalyst, as an alkylation catalyst, as an isomerization catalyst, or as a catalyst in the conversion of alcohols to olefins, and more preferably in the conversion of oxygenates to olefins.

It is preferred that the zeolitic material is used in a methanol-to-olefin process (MTO process), in a dimethylether to olefin process (DTO process), methanol-to-gasoline process (MTG process), in a methanol-to-hydrocarbon process, in a methanol to aromatics process, in a biomass to olefins and/or biomass to aromatics process, in a methane to benzene process, for alkylation of aromatics, or in a fluid catalytic cracking process (FCC process), more preferably in a methanolto-olefin process (MTO process) and/or in a dimethylether to olefin process (DTO process), and more preferably in a methanol-to-propylene process (MTP process), in a methanol-topropylene/butylene process (MT3/4 process), in a dimethylether-to-propylene process (DTP process), in a dimethylether-to-propylene/butylene process (DT3/4 process), and/or in a dimethylether-to-ethylene/propylene (DT2/3 process).

The unit bar(abs) refers to an absolute pressure of 105 Pa and the unit Angstrom refers to a length of 10-¹⁰ m.

The present invention is further illustrated by the following set of embodiments and combinations of embodiments resulting from the dependencies and back-references as indicated. In particular, it is noted that in each instance where a range of embodiments is mentioned, for example in the context of a term such as “The zeolitic material of any one of embodiments 1 to 4”, every embodiment in this range is meant to be explicitly disclosed for the skilled person, i.e. the wording of this term is to be understood by the skilled person as being synonymous to “The zeolitic material of any one of embodiments 1, 2, 3, and 4”. Further, it is explicitly noted that the following set of embodiments is not the set of claims determining the extent of protection, but represents a suitably structured part of the description directed to general and preferred aspects of the present invention.
1. A zeolitic material having the ITH framework structure type, preferably obtainable and/or obtained according to the process of any one of embodiments 21 to 59, wherein the zeolitic material comprises YO₂ and optionally X₂O₃ in its framework structure, wherein Y is a tetravalent element and X is a trivalent element, wherein the framework structure of the zeolitic material comprises less than 4 weight-% of Ge calculated as GeO₂ and based on 100 weight-% of YO₂ contained in the framework structure,
wherein the zeolitic material comprises less than 1.5 weight-% of B calculated as B₂03 and based on 100 weight-% of X₂O₃ contained in the framework structure, and wherein the zeolitic material has a molar ratio YO₂: X₂O₃ of equal or greater than 50.
2. The zeolitic material of embodiment 1, wherein the zeolitic material comprises YO₂ and X₂O₃ in its framework structure, wherein the zeolitic material has a molar ratio YO₂: X₂03 of equal or greater than 60, wherein the zeolitic material preferably has a molar ratio YO₂: X₂O₃ of equal or greater than 100, more preferably in the range of from 100 to 250, more preferably in the range of from 105 to 225, more preferably in the range of from 110 to 200, more preferably in the range of from 120 to 150, more preferably in the range of from 135 to 145.
3. The zeolitic material of embodiment 1 or 2, wherein the framework structure of the zeolitic material comprises less than 3 weight-% of Ge calculated as GeO₂ and based on 100 weight-% of YO₂ contained in the framework structure, preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%.
4. The zeolitic material of any one of embodiments 1 to 3, wherein the zeolitic material comprises less than 3 weight-% of B calculated as B₂O₃ and based on 100 weight-% of X₂03 contained in the framework structure, preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%.
5. The zeolitic material of any one of embodiments 1 to 4, wherein Y is selected from the group consisting of Si, Sn, Ti, Zr, and mixtures of two or more thereof, Y preferably being Si and/or Ti, wherein Y is more preferably Si.
6. The zeolitic material of any one of embodiments 1 to 5, wherein X is selected from the group consisting of Al, In, Ga, Fe, and mixtures of two or more thereof, X preferably being Al and/or Ga, wherein X is more preferably Al.
7. The zeolitic material of any one of embodiments 1 to 6, wherein Y comprises, preferably consists of, Si, wherein the ²⁹Si MAS NMR of the zeolitic material comprises:
   a first peak having a maximum in the range of from −101.0 to −107.0 ppm, preferably of from −102.0 to −106.5 ppm, more preferably of from −103.0 to −106.2 ppm, more preferably of from −104.0 to −106.0 ppm, more preferably of from −105.0 to −105.7 ppm, and more preferably of from −105.3 to −105.5 ppm;
   a second peak having a maximum in the range of from −105.0 to −112.7 ppm, preferably of from −106.5 to −112.2 ppm, more preferably of from −107.5 to −111.0 ppm, more preferably of from −110.0 to −111.7 ppm, more preferably of from −111.0 to −111.6 ppm, and more preferably of from −111.2 to −111.4 ppm;
      a third peak having a maximum in the range of from −111.0 to −116.0 ppm, preferably of from −112.0 to −115.5 ppm, more preferably of from −113.0 to −115.2 ppm, more preferably of from −113.5 to −115.0 ppm, more preferably of from −114.1 to −114.7 ppm, and more preferably of from −114.3 to −114.5 ppm; and
   a fourth peak having a maximum in the range of from −115.1 to −118.4 ppm, preferably of from −115.6 to −117.9 ppm, more preferably of from −116.1 to −117.4 ppm, more preferably of from −116.4 to −117.1 ppm, and more preferably of from −116.6 to −116.9 ppm,

wherein preferably the ²⁹Si MAS NMR of the zeolitic material comprises only four peaks in the range of from −80 to −130 ppm,

wherein the ²⁹Si MAS NMR of the zeolitic material is preferably determined according to reference example 6.
8. The zeolitic material of any one of embodiments 1 to 7, wherein the zeolitic material comprises F, wherein the ¹⁹F MAS NMR of the zeolitic material comprises:
   a first peak having a maximum in the range of from −32 to −38 ppm, preferably in the range of from −33.0 to −37.4 ppm, more preferably in the range of from −34.0 to −36.0 ppm, more preferably in the range of from −35.0 to −36.0 ppm,
   a second peak having a maximum in the range of from −61.3 to −66.3 ppm, preferably in the range of from −61.0 to −65.8 ppm, more preferably of from −62.3 to −65.3 ppm, more preferably of from −62.8 to −64.8 ppm, more preferably of from −63.3 to −64.3 ppm; wherein preferably the ¹⁹F MAS NMR of the zeolitic material comprises only two peaks in the range of from 0 to −100 ppm,
wherein the ¹⁹F MAS NMR of the zeolitic material is preferably determined according to reference example 6.
9. The zeolitic material of any one of embodiments 1 to 8, wherein the zeolitic material comprises X₂O₃ in its framework structure, wherein X comprises, preferably consists of, Al, wherein the ²⁷AI MAS NMR of the zeolitic material comprises:
   a peak having a maximum in the range of from 50 to 58 ppm, preferably of from 51 to 57 ppm, more preferably of from 52 to 56 ppm, more preferably of from 52.5 to 55.5 ppm, more preferably of from 53 to 55 ppm, wherein preferably the ²⁷AI MAS NMR of the zeolitic material comprises a single peak having a maximum in the range of from −40 to 140 ppm, wherein the ²⁷AI MAS NMR of the zeolitic material is preferably determined according to reference example 6.
10. The zeolitic material of any one of embodiments 1 to 9, wherein the zeolitic material, preferably the calcined zeolitic material, displays an X-ray powder diffraction pattern comprising at least the following reflections:

| | Diffraction angle 2θ/° |
|---|---|
| Intensity (%) | [Cu K(alpha 1)] |
| 100 | [7.00-7.20] |
| [33-53] | [7.82-8.02] |
| [22-42] | [8.60-8.80] |
| [42-62] | [11.12-11.32] |
| [28-48] | [15.38-15.58] |
| [19-39] | [16.05-16.25] |
| [19-39] | [16.48-16.68] |
| [17-37] | [20.94-21.14] |
| [14-34] | [21.40-21.60] |
| [77-97] | [22.92-23.12] |
| [57-77] | [23.54-23.74] |
| [31-51] | [23.90-24.10] |

wherein 100% relates to the intensity of the maximum peak in the X-ray powder diffraction pattern, and wherein the X-ray powder diffraction pattern is preferably determined according to reference example 2 disclosed herein,
wherein preferably the zeolitic material, more preferably the calcined zeolitic material, displays an X-ray powder diffraction pattern comprising at least the following reflections:

| | Diffraction angle 2θ/° |
|---|---|
| Intensity (%) | [Cu K(alpha 1)] |
| 100 | [7.05-7.15] |
| [33-53] | [7.87-7.97] |
| [22-42] | [8.65-8.75] |
| [42-62] | [11.17-11.27] |
| [28-48] | [15.43-15.53] |
| [19-39] | [16.10-16.20] |
| [19-39] | [16.53-16.63] |
| [17-37] | [20.99-21.09] |
| [14-34] | [21.45-21.55] |
| [77-97] | [22.97-23.07] |
| [57-77] | [23.59-23.69] |
| [31-51] | [23.95-24.05] |

wherein 100% relates to the intensity of the maximum peak in the X-ray powder diffraction pattern, and wherein the X-ray powder diffraction pattern is preferably determined according to reference example 2 disclosed herein.
11. The zeolitic material of any one of embodiments 1 to 9, wherein the zeolitic material, preferably the calcined zeolitic material, displays an X-ray powder diffraction pattern comprising at least the following reflections:

| | Diffraction angle 2θ/° |
|---|---|
| Intensity (%) | [Cu K(alpha 1)] |
| [24-44] | [8.09-8.29] |
| [25-45] | [11.29-11.49] |
| [20-40] | [16.08-16.28] |
| [33-53] | [21.13-21.33] |
| [28-48] | [21.51-21.71] |
| [24-44] | [22.45-22.65] |
| 100 | [23.10-23.30] |
| [67-87] | [23.77-23.97] |
| [44-64] | [24.19-24.39] |
| [18-38] | [27.76-27.96] |

wherein 100% relates to the intensity of the maximum peak in the X-ray powder diffraction pattern, and wherein the X-ray powder diffraction pattern is preferably determined according to reference example 2 disclosed herein, wherein preferably the zeolitic material, more preferably the calcined zeolitic material, displays an X-ray powder diffraction pattern comprising at least the following reflections:

| | Diffraction angle 2θ/° |
|---|---|
| Intensity (%) | [Cu K(alpha 1)] |
| [29-39] | [8.14-8.24] |
| [30-40] | [11.34-11.44] |
| [25-35] | [16.13-16.23] |
| [38-48] | [21.18-21.28] |
| [33-43] | [21.56-21.66] |
| [29-39] | [22.50-22.60] |
| 100 | [23.15-23.25] |
| [72-82] | [23.82-23.92] |
| [49-59] | [24.24-24.34] |
| [23-33] | [27.81-27.91] |

wherein 100% relates to the intensity of the maximum peak in the X-ray powder diffraction pattern, and wherein the X-ray powder diffraction pattern is preferably determined according to reference example 2 disclosed herein.
12. The zeolitic material of any one of embodiments 1 to 11, wherein the BET surface area of the zeolitic material is in the range of from 50 to 800 m²/g, preferably from 100 to 700 m²/g, more preferably from 200 to 600 m²/g, more preferably from 300 to 500 m²/g, more preferably from 350 to 450 m²/g, more preferably from 375 to 425 m²/g, more preferably from 390 to 410 m²/g, more preferably from 395 to 405 m²/g, wherein preferably the BET surface area is determined according to ISO 9277:2010.
13. The zeolitic material of any one of embodiments 1 to 12, wherein the micropore volume of the zeolitic material is in the range of from 0.05 to 0.5 cm³/g, preferably from 0.075 to 0.3 cm³/g, more preferably from 0.1 to 0.25 cm³/g, more preferably from 0.11 to 0.19 cm³/g, more preferably from 0.13 to 0.17 cm³/g, and more preferably from 0.14 to 0.16 cm³/g, wherein preferably the micropore volume is determined according to ISO 15901-1:2016.
14. The zeolitic material of any one of embodiments 1 to 13, wherein the mesopore volume of the zeolitic material is in the range of from 0.05 to 0.5 cm³/g, preferably from 0.1 to 0.3 cm³/g, more preferably from 0.18 to 0.26 cm³/g, more preferably from 0.20 to 0.24 cm³/g, and more preferably from 0.21 to 0.23 cm³/g, wherein preferably the mesopore volume is determined according to ISO 15901-3:2007.
15. The zeolitic material of any one of embodiments 1 to 14, having a nanosheet-like crystal morphology, wherein preferably the thickness of a nanosheet is in the range of from 5 to 100 nm, preferably in the range of from 10 to 50 nm, more preferably in the range of from 25 to 35 nm, preferably determined according to reference example 4 and/or according to reference example 5.
16. The zeolitic material of any one of embodiments 1 to 15, showing in the temperature programmed desorption of ammonia (NH₃-TPD) a first desorption peak centered in the range of from 170 to 200° C., preferably in the range of from 180 to 190° C., more preferably in the range of from 183 to 187° C., and a second desorption peak centered in the range of from 370 to 410° C., preferably in the range of from 380 to 400° C., more preferably in the range of from 385 to 395° C., preferably determined according to reference example 7.
17. The zeolitic material of any one of embodiments 1 to 16, wherein the zeolitic material comprises one or more metal cations M at the ion-exchange sites of the framework structure of the zeolitic material, wherein the one or more metal cations M are preferably selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, preferably selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, more preferably from the group consisting of Sr, Cr, Mg, Ca, Mo, Fe, Co, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Cr, Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, and more preferably from the group consisting of Fe, Cu, Mg, Ca, Zn, Mo, La, Ce, Pr, Nd, Sm, Eu, Gd, Tb, Dy, Ho, Er, Tm, Yb, Lu, Y, Sc, and mixtures of two or more thereof.
18. The zeolitic material of embodiment 17, wherein the zeolitic material comprises the one or more metal cations M in an amount in the range of from 0.01 to 10 weight-% based on 100 weight-% of Si in the zeolitic material calculated as SiO₂, preferably in the range of from 0.05 to 7 weight-%, more preferably in the range of from 0.1 to 5 weight-%, more preferably in the range of from 0.5 to 4.5 weight-%, more preferably in the range of from 1 to 4 weight-%, more preferably in the range of from 1.5 to 3.5 weight-%.
19. The zeolitic material of any one of embodiments 1 to 18, wherein from 95 to 100 weight-% of the zeolitic material consists of Si, optionally Al, O, H, and the one or more metal cations M, calculated based on the total weight of the zeolitic material, preferably from 97 to 100 weight-%, more preferably from 99 to 100 weight-%.
20. The zeolitic material of any one of embodiments 1 to 19, wherein from 95 to 100 weight-% of the framework of the zeolitic material consists of Si, optionally Al, O, and H, based on the total weight of the framework of the zeolitic material, preferably from 97 to 100 weight-%, more preferably from 99 to 100 weight-%.
21. A process for the preparation of a zeolitic material having the ITH framework structure type, preferably of a zeolitic material according to any one of embodiments 1 to 20, wherein the process comprises
   (1) preparing a mixture comprising one or more organotemplates as structure directing agents, one or more sources of YO₂, optionally one or more sources of X₂03, seed crystals, and a solvent system, wherein Y is tetravalent element and X is a trivalent element;
   (2) heating the mixture obtained in (1) for crystallizing a zeolitic material having the ITH framework structure type comprising YO₂ and optionally X₂O₃ in its framework structure;
wherein the one or more organotemplates comprise a polymeric cation comprising a unit of formula (I):

[R¹R²N⁺−R⁵−N+R³R⁴−R⁶]ₙ (I);

wherein R¹, R², R³, and R⁴ independently from one another is (C₁-C₄)alkyl, preferably (C₁-C₃)alkyl, more preferably ethyl or methyl, and more preferably methyl;
wherein R⁵ is selected from the group consisting of tetramethylene, pentamethylene, hexamethylene, and heptamethylene, wherein preferably R⁵ is pentamethylene or hexamethylene, wherein more preferably R⁵ is hexamethylene;
wherein R⁶ is selected from the group consisting of trimethylene, tetramethylene, and pentamethylene, wherein preferably R⁶ is trimethylene or tetramethylene, wherein more preferably R⁶ is tetramethylene;
wherein n is a natural number in the range of from 1 to 50, preferably in the range of from 2 to 40, more preferably in the range of from 5 to 30, more preferably in the range of from 10 to 23, more preferably in the range of from 11 to 22.
22. The process of embodiment 21, wherein the organotemplate: YO₂ molar ratio of the one or more organotemplates to the one or more sources of YO₂ calculated as YO₂ in the mixture prepared in (1) and heated in (2) is in the range of from 0.001 to 0.5, preferably from 0.0012 to 0.27, more preferably from 0.0015 to 0.24, more preferably from 0.002 to 0.2, more preferably from 0.0025 to 0.1, more preferably from 0.003 to 0.02, more preferably from 0.0035 to 0.015, more preferably from 0.004 to 0.01, and more preferably from 0.0045 to 0.006.
23. The process of embodiment 21 or 22, wherein the one or more organotemplates are provided as salts, preferably as one or more salts selected from the group consisting of halides, sulfate, nitrate, phosphate, acetate, hydroxide, and mixtures of two or more thereof, more preferably from the group consisting of bromide, chloride, hydroxide, sulfate, and mixtures of two or more thereof, wherein more preferably the one or more organotemplates are provided as hydroxides and/or bromides, and more preferably as hydroxides.
24. The process of any one of embodiments 21 to 23, wherein Y is selected from the group consisting of Si, Sn, Ti, Zr, Ge, and mixtures of two or more thereof, Y preferably being Si and/or Ti, wherein Y is more preferably Si.
25. The process of any one of embodiments 21 to 24, wherein X is selected from the group consisting of Al, B, In, Ga, and mixtures of two or more thereof, preferably from the group consisting of Al, B, Ga, and mixtures of two or more thereof, X more preferably being Al and/or B, wherein X is more preferably Al.
26. The process of any one of embodiments 21 to 25, wherein the seed crystals comprise one or more zeolitic materials having the ITH framework structure type, wherein preferably the seed crystals comprise ITQ-13, wherein more preferably the seed crystals consist of one or more zeolitic materials having the ITH framework structure type, wherein more preferably the seed crystals consist of ITQ-13.
27. The process of any one of embodiments 21 to 26, wherein the seed crystals comprise one or more zeolitic materials having the ITH framework structure type, preferably one or more zeolitic materials according to any one of embodiments 1 to 20 and 60, wherein more preferably the seed crystals consist of one or more zeolitic materials having the ITH framework structure type, wherein more preferably the seed crystals consist of one or more zeolitic materials according to any one of embodiments 1 to 20 and 60.
28. The process of any one of embodiments 21 to 27, wherein the seed crystals comprise one or more zeolitic materials having the ITH framework structure type, preferably one or more zeolitic materials having the ITH framework structure type, wherein from 95 to 100 weight-% of the one or more zeolitic materials having the ITH framework structure type consist of Si, 0, and H, more preferably from 97 to 100 weight-%, more preferably from 99 to 100 weight-%.
29. The process of any one of embodiment 21 to 28, wherein the amount of seed crystals comprised in the mixture prepared in (1) is in the range of from 0.1 to 15 weight-% based on 100 weight-% of the one or more sources of YO₂ calculated as YO₂, preferably from 0.5 to 12 weight-%, more preferably from 1 to 10 weight-%, more preferably from 2 to 8 weight-%, more preferably from 3 to 7 weight-%, more preferably from 5 to 6 weight-%.
30. The process of any one of embodiments 21 to 29, wherein the mixture prepared in (1) and heated in (2) contains less than 5 weight-% of Ge calculated as GeO₂ and based on 100 weight-% of the one or more sources of YO₂ calculated as YO₂, preferably less than 3 weight-%, more preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%.
31. The process of any one of embodiments 21 to 30, wherein the mixture prepared in (1) and heated in (2) contains less than 5 weight-% of B calculated as B₂O₃ and based on 100 weight-% of the one or more sources of X₂O₃ calculated as X₂03, preferably less than 3 weight-%, more preferably less than 1 weight-%, more preferably less than 0.5 weight-%, more preferably less than 0.1 weight-%, more preferably less than 0.05 weight-%, more preferably less than 0.01 weight-%, more preferably less than 0.005 weight-%, and more preferably less than 0.001 weight-%.
32. The process of any one of embodiments 21 to 31, wherein the mixture comprises one or more sources for X₂03, wherein the X₂O₃: YO₂ molar ratio of the one or more sources of X₂O₃ calculated as X₂O₃ to the one or more sources of YO₂ calculated as YO₂ in the mixture prepared in (1) and heated in (2) is in the range of from 0.001 to 0.1, preferably of from 0.0015 to 0.05, more preferably of from 0.0017 to 0.030, more preferably of from 0.0019 to 0.015, more preferably of from 0.002 to 0.01, more preferably of from 0.0025 to 0.007.
33. The process of any one of embodiments 21 to 32, wherein the mixture prepared in (1) further comprises one or more sources of fluoride, wherein preferably the fluoride: YO₂ molar ratio of the one or more sources of fluoride calculated as the element to the one or more sources of YO₂ calculated as YO₂ in the mixture prepared in (1) and heated in (2) is in the range of from 0.01 to 2, preferably from 0.05 to 1.5, more preferably from 0.1 to 1, more preferably from 0.13 to 0.55, more preferably from 0.14 to 0.45, more preferably from 0.15 to 0.4, more preferably from 0.2 to 0.3.
34. The process of embodiment 33, wherein the one or more sources of fluoride is selected from fluoride salts, HF, and mixtures of two or more thereof, preferably from the group consisting of alkali metal fluoride salts, ammonium fluoride salts, HF, and mixtures of two or more thereof, wherein more preferably the one or more sources of fluoride comprise HF or ammonium fluoride, wherein more preferably the one or more sources of fluoride comprise HF, wherein more preferably the one or more sources of fluoride consist of HF.
35. The process of any one of embodiments 21 to 34, wherein the one or more sources for YO₂ comprises one or more compounds selected from the group consisting of fumed silica, silica hydrosols, reactive amorphous solid silicas, silica gel, silicic acid, water glass, sodium metasilicate hydrate, sesquisilicate, disilicate, colloidal silica, silicic acid esters, and mixtures of two or more thereof, preferably from the group consisting of fumed silica, silica hydrosols, silica gel, silicic acid, water glass, sodium metasilicate hydrate, sesquisilicate, disilicate, colloidal silica, tetra(C₁-C₄)alkylorthosilicate, and mixtures of two or more thereof, more preferably from the group consisting of fumed silica, silica hydrosols, silicic acid, tetra(C₂-C₃)alkylorthosilicate, and mixtures of two or more thereof, wherein more preferably the one or more sources for YO₂ fumed silica, wherein more preferably the one or more sources for YO₂ consist of fumed silica.
36. The process of any one of embodiments 21 to 35, wherein the one or more sources for X₂O₃ comprises one or more compounds selected from the group consisting of alumina, aluminates, aluminum salts, and mixtures of two or more thereof, preferably from the group consisting of alumina, aluminum salts, and mixtures of two or more thereof, more preferably from the group consisting of alumina, aluminum tri(C-C₅)alkoxide, AIO(OH), AI(OH)₃, aluminum halides, preferably aluminum fluoride and/or chloride and/or bromide, more preferably aluminum fluoride and/or chloride, and even more preferably aluminum chloride, aluminum sulfate, aluminum phosphate, aluminum fluorosilicate, and mixtures of two or more thereof, more preferably from the group consisting of aluminum tri(C2-C₄)alkoxide, AIO(OH), AI(OH)₃, aluminum chloride, aluminum sulfate, aluminum phosphate, and mixtures of two or more thereof, more preferably from the group consisting of aluminum tri(C₂-C₃)alkoxide, AIO(OH), AI(OH)₃, aluminum chloride, aluminum sulfate, and mixtures of two or more thereof, more preferably from the group consisting of aluminum tripropoxides, AIO(OH), aluminum sulfate, and mixtures of two or more thereof, wherein more preferably the one or more sources for X₂O₃ comprises AIO(OH), and wherein more preferably the one or more sources for X₂O₃ consist of AIO(OH), preferably gammaAIO(OH).
37. The process of any one of embodiments 21 to 35, wherein the one or more sources for X₂O₃ comprises a zeolitic material comprising YO₂ and X₂O₃ in its framework structure, wherein Y is tetravalent element and X is a trivalent element;
wherein Y is preferably selected from the group consisting of Si, Sn, Ti, Zr, Ge, and mixtures of two or more thereof, Y more preferably being Si and/or Ti, more preferably Si; wherein X is preferably selected from the group consisting of Al, B, In, Ga, and mixtures of two or more thereof, more preferably from the group consisting of Al, B, Ga, and mixtures of two or more thereof, X more preferably being Al and/or B, more preferably Al; wherein the zeolitic material has a molar ratio YO₂: X₂O₃ of equal or greater than 0.1, preferably in the range of from 0.3 to 100, more preferably in the range of from 0.5 to 50, more preferably in the range of from 0.7 to 10, more preferably in the range of from 0.9 to 5, more preferably in the range of from 1 to 3;
wherein the zeolitic material preferably has a framework structure type selected from the group consisting of FAU, GIS, MOR, LTA, FER, TON, MTT, BEA, MEL, MWW, MFS, MFl, mixed structures of two or more thereof, and a mixture of two or more thereof, more preferably selected from the group consisting of FAU, GIS, MOR, LTA, FER, TON, MTT, BEA, MEL, MWW, MFS, MFl, mixed structures of two or more thereof, and a mixture of two or more thereof, more preferably an FAU and/or a LTA framework structure type.
38. The process of embodiment 37, wherein the zeolitic material having an LTA-type framework structure type is selected from the group consisting of Linde Type A (zeolite A), Alpha, [AI—Ge-O]-LTA, N-A, LZ-215, SAPO-42, ZK-4, ZK-21, Dehyd. Linde Type A (dehyd. zeolite A), ZK-22, ITQ-29, UZM-9, including mixtures of two or more thereof, preferably from the group consisting of Linde Type A, Alpha, N-A, LZ-215, SAPO-42, ZK-4, ZK-21, Dehyd. Linde Type A, ZK-22, ITQ-29, UZM-9, including mixtures of two or more thereof, more preferably from the group consisting of Linde Type A, Alpha, N-A, LZ-215, ZK-4, ZK-21, Dehyd. Linde Type A, ZK-22, ITQ-29, UZM-9, including mixtures of two or more thereof, more preferably from the group consisting of Linde Type A, Alpha, N-A, LZ-215, ZK-4, ZK-21, ZK-22, ITQ-29, UZM-9, including mixtures of two or more thereof.
39. The process of embodiment 37, wherein the zeolitic material having an FAU framework structure type is selected from the group consisting of ZSM-3, Faujasite, [AI—Ge-O]-FAU, CSZ-1, ECR-30, Zeolite X, Zeolite Y, LZ-210, SAPO-37, ZSM-20, Na—X, US-Y, Na—Y, [Ga—Ge-O]-FAU, Li-LSX, [Ga-AI—Si-O]-FAU, [Ga—Si-O]-FAU, and a mixture of two or more thereof, preferably from the group consisting of ZSM-3, Faujasite, CSZ-1, ECR-30, Zeolite X, Zeolite Y, LZ-210, ZSM-20, Na—X, US-Y, Na—Y, Li-LSX, and a mixture of two or more thereof, more preferably from the group consisting of Faujasite, Zeolite X, Zeolite Y, Na—X, US-Y, Na—Y, and a mixture of two or more thereof, more preferably from the group consisting of Faujasite, Zeolite X, Zeolite Y, and a mixture of two or more thereof, wherein more preferably the zeolitic material having an FAU framework structure type comprises Zeolite X and/or Zeolite Y, preferably Zeolite X, wherein more preferably the zeolitic material having an FAU framework structure type is Zeolite X and/or Zeolite Y, preferably Zeolite X.
40. The process of any one of embodiments 21 to 39, wherein the solvent system is selected from the group consisting of optionally branched (C₁-C₄)alcohols, distilled water, and mixtures thereof, preferably from the group consisting of optionally branched (C₁-C₃)alcohols, distilled water, and mixtures thereof, more preferably from the group consisting of methanol, ethanol, distilled water, and mixtures thereof, wherein more preferably the solvent system comprises distilled water, wherein more preferably the solvent system consists of distilled water.
41. The process of embodiment 40, wherein the H₂O: YO₂ molar ratio of H₂O to the one or more sources of YO₂ calculated as YO₂ in the mixture prepared in (1) and heated in (2) is in the range of from 0.1 to 15, preferably from 0.2 to 7.5, more preferably from 0.4 to 5, more preferably from 0.5 to 4, more preferably from 0.9 to 3.1, more preferably from 1 to 3.
42. The process of any one of embodiments 21 to 41, wherein heating in (2) is conducted for a duration in the range of from 10 min to 35 d, preferably of from 1 h to 30 d, more preferably from 2 d to 25 d, more preferably from 5 d to 20 d, more preferably from 6 d to 15 d, more preferably from 7 d to 13 d, more preferably from 9 d to 11 d, and more preferably from 9.5 to 10.5 d.
43. The process of any one of embodiments 21 to 42 wherein heating in (2) is conducted at a temperature in the range of from 80 to 220° C., preferably of from 110 to 200° C., more preferably of from 130 to 190° C., more preferably of from 140 to 180° C., more preferably from 145 to 175° C., more preferably of from 150 to 170° C., and more preferably of from 155 to 165° C.
44. The process of any one of embodiments 21 to 43, wherein heating in (2) is conducted under autogenous pressure, preferably under solvothermal conditions, more preferably under hydrothermal conditions, wherein preferably heating in (2) is performed in a pressure tight vessel, preferably in an autoclave.
45. The process of any one of embodiments 21 to 44, wherein the one or more organotemplates are prepared according to a process comprising
   (a) preparing a reaction mixture comprising a compound having the formula (II)

R¹R²N⁺−R⁵−N+R³R⁴ (II)
a compound having the formula (III)

Rₐ−R⁶−R_{b} (III)
and a solvent system, to obtain a reaction mixture;
(b) heating the reaction mixture, to obtain a mixture comprising one or more organotemplates;
wherein R¹, R², R³, and R⁴ independently from one another is (C₁-C₄)alkyl, preferably (C₁-C₃)alkyl, more preferably ethyl or methyl, and more preferably methyl;
wherein R⁵ is selected from the group consisting of tetramethylene, pentamethylene, hexamethylene, and heptamethylene, wherein preferably R⁵ is pentamethylene or hexamethylene, wherein more preferably R⁵ is hexamethylene;
wherein R⁶ is selected from the group consisting of trimethylene, tetramethylene, and pentamethylene, wherein preferably R⁶ is trimethylene or tetramethylene, wherein more preferably R⁶ is tetramethylene; and
wherein Rₐ and R_{b} independently from each other is selected from the group consisting of F, C1, Br, I, tosyl (OTs), mesyl, triflourmethansulfonate (OTf), and OH, preferably from the group consisting of F, C1, Br, I, and OH, more preferably from the group consisting of Br, I, and OH, more preferably Rₐ and R_{b} independently from each other is Br.
46. The process of embodiment 45, wherein a molar ratio of the compound having the formula (II) to the compound having the formula (III) in the mixture in (a) is in the range of from 0.1:1 to 10:1, preferably in the range of from 0.5:1 to 2:1, more preferably in the range of from 0.9:1 to 1.1:1.
47. The process of embodiment 45 or 46, wherein heating in (b) is conducted of reflux of the solvent system, wherein preferably heating in (b) is conducted at a temperature in the range of from 50 to 110° C., preferably in the range of from 70 to 90° C., more preferably in the range of from 75 to 85° C.
48. The process of any one of embodiments 45 to 47, wherein heating in (b) is conducted for a duration in the range of from 1 to 25 h, preferably from 9 to 15 h, more preferably from 11 to 13 h.
49. The process of any one of embodiments 45 to 48, wherein the solvent system comprises one or more of water, methanol, ethanol, propanol, and tetrahydrofuran, preferably one or more of methanol, ethanol, and propanol, more preferably ethanol, wherein more preferably the solvent system consists of ethanol.
50. The process of any one of embodiments 45 to 49, wherein the process further comprises
   (c) isolating one or more organotemplates from the mixture obtained in (b), and/or
   (d) washing the one or more organotemplates obtained in (b) or (c).
51. The process of embodiment 50, wherein isolating in (c) is conducted by filtration.
52. The process of embodiment 50 or 51, wherein washing in (d) is conducted with one or more of diethylether, tetrahydrofuran, and ethyl acetate, preferably with diethyl ether.
53. The process of any one of embodiments 21 to 52, wherein the process further comprises
   (3) isolating the zeolitic material obtained in (2), and/or
   (4) washing the zeolitic material obtained in (2) or (3), and/or
   (5) drying the zeolitic material obtained in (2), (3), or (4), in a gas atmosphere, and/or
   (6) calcining the zeolitic material obtained in (2), (3), (4) or (5) in a gas atmosphere, and/or
   (7) subjecting the zeolitic material obtained in (2), (3), (4), (5) or (6) to an ion-exchange procedure with one or more metal cations M,
wherein the steps (3) and/or (4) and/or (5) and/or (6) and/or (7) can be conducted in any order, and
wherein one or more of said steps is preferably repeated one or more times.
54. The process of embodiment 53, wherein the one or more metal cations M are selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, preferably selected from the group consisting of Sr, Zr, Cr, Mg, Ca, Mo, Fe, Co, Ni, Cu, Zn, Ru, Rh, Pd, Ag, Os, Ir, Pt, Au, La, Ce, Pr, Nd, Sm, Eu, Gd, Tb, Dy, Ho, Er, Tm, Yb, Lu, Y, Sc, and mixtures of two or more thereof, more preferably from the group consisting of Sr, Cr, Mg, Ca, Mo, Fe, Co, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Cr, Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, more preferably from the group consisting of Mg, Ca, Mo, Fe, Ni, Cu, Zn, Ag, La, Ce, Pr, Nd, Sm, Eu, Gd, Tb, Dy, Ho, Er, Tm, Yb, Lu, Y, Sc, and mixtures of two or more thereof, and more preferably from the group consisting of Fe, Cu, Mg, Ca, Zn, Mo, La, Ce, Pr, Nd, Sm, Eu, Gd, Tb, Dy, Ho, Er, Tm, Yb, Lu, Y, Sc, and mixtures of two or more thereof, wherein the one or more metal cations M are located at the ion-exchange sites of the framework structure of the zeolitic material.
55. The process of embodiment 53 or 54, wherein drying in (5) is conducted at a temperature of the gas atmosphere in the range of from 60 to 140° C., preferably of from 80 to 120° C., and more preferably of from 90 to 110° C.
56. The process of any one of embodiments 53 to 55, wherein the gas atmosphere for drying in (5) comprises nitrogen, oxygen, or a mixture thereof, wherein the gas atmosphere is preferably oxygen, air, or lean air.
57. The process of any one of embodiments 53 to 56, wherein calcination in (6) is conducted for a duration in the range of from 0.5 to 15 h, preferably of from 1 to 10 h, more preferably of from 2 to 8 h, more preferably of from 3 to 7 h, more preferably of from 3.5 to 6.5 h, more preferably of from 4 to 6 h, more preferably of from 4.5 to 5.5 h.
58. The process of any one of embodiments 53 to 57, wherein the gas atmosphere for calcination in (6) comprises nitrogen, oxygen, or a mixture thereof, wherein the gas atmosphere is preferably oxygen, air, or lean air.
59. The process of any one of embodiments 53 to 58, wherein calcination in (6) is conducted at a temperature of the gas atmosphere in the range of from 300 to 800° C., preferably of from 375 to 725° C., more preferably of from 425 to 675° C., more preferably of from 475 to 625° C., and more preferably of from 525 to 575° C.
60. A zeolitic material having the ITH framework structure type obtainable and/or obtained from the process of any one of embodiments 21 to 59.
61. A method for the conversion of oxygenates to olefins comprising
   (i) providing a catalyst according to any one of embodiments 1 to 20 and 60;
   (ii) providing a gas stream comprising one or more oxygenates and optionally one or more olefins and/or optionally one or more hydrocarbons;
   (iii) contacting the catalyst provided in (i) with the gas stream provided in (ii) and converting one or more oxygenates to one or more olefins and optionally to one or more hydrocarbons;
   (iv) optionally recycling one or more of the one or more olefins and/or of the one or more hydrocarbons contained in the gas stream obtained in (iii) to (ii).
62. The method of embodiment 61, wherein the catalyst is provided in a fixed bed or in a fluidized bed.
63. The method of embodiment 61 or 62, wherein the gas stream provided in (ii) comprises one or more oxygenates selected from the group consisting of aliphatic alcohols, ethers, carbonyl compounds and mixtures of two or more thereof, preferably from the group consisting of (C1-C₆) alcohols, di(C₁-C₃)alkyl ethers, (C1-C₆) aldehydes, (C₂-C₆) ketones and mixtures of two or more thereof, more preferably consisting of (C1-C₄) alcohols, di(C₁-C₂)alkyl ethers, (C1-C₄) aldehydes, (C₂-C₄) ketones and mixtures of two or more thereof, more preferably from the group consisting of methanol, ethanol, n-propanol, isopropanol, butanol, dimethyl ether, diethyl ether, ethyl methyl ether, diisopropyl ether, di-n-propyl ether, formaldehyde, dimethyl ketone and mixtures of two or more thereof, more preferably from the group consisting of methanol, ethanol, dimethyl ether, diethyl ether, ethyl methyl ether and mixtures of two or more thereof, the gas stream more preferably comprising methanol and/or dimethyl ether, more preferably methanol.
64. The method of any one of embodiments 61 to 63, wherein the content of oxygenates in the gas stream provided in (ii) is in the range of from 2 to 100% by volume based on the total volume, preferably from 3 to 99% by volume, more preferably from 4 to 95% by volume, more preferably from 5 to 80% by volume, more preferably from 6 to 50% by volume.
65. The method of any one of embodiments 61 to 64, wherein the gas stream provided in (ii) comprises water, wherein the water content in the gas stream provided in (ii) is preferably in the range from 5 to 60% by volume, more preferably from 10 to 50% by volume.
66. The method of any one of embodiments 61 to 65, wherein the gas stream provided in (ii) further comprises one or more diluting gases, preferably one or more diluting gases in an amount ranging from 0.1 to 90% by volume, more preferably from 1 to 85% by volume, more preferably from 5 to 80% by volume, more preferably from 10 to 75% by volume.
67. The method of any one of embodiments 61 to 66, wherein the one or more diluting gases are selected from the group consisting of H₂O, helium, neon, argon, krypton, nitrogen, carbon monoxide, carbon dioxide, and mixtures of two or more thereof, preferably from the group consisting of H₂O, argon, nitrogen, carbon dioxide, and mixtures of two or more thereof, wherein more preferably the one or more diluting gases comprise H₂O, wherein more preferably the one or more diluting gases is H₂O.
68. The method of any one of embodiments 61 to 67, wherein the contacting according to (iii) is effected at a temperature in the range from 225 to 700° C., preferably from 275 to 650° C., more preferably from 325 to 600° C., more preferably from 375 to 550° C., more preferably from 425 to 525° C., and more preferably from 450 to 500° C.
69. The method of any one of embodiments 61 to 68, wherein the contacting according to (iii) is effected at a pressure in the range from 0.01 to 25 bar, preferably from 0.1 to 20 bar, more preferably from 0.25 to 15 bar, more preferably from 0.5 to 10 bar, more preferably from 0.75 to 5 bar, more preferably from 0.8 to 2 bar, more preferably from 0.85 to 1.5 bar, more preferably from 0.9 to 1.1 bar.
70. The method of any one of embodiments 61 to 69, wherein the method is a continuous method, wherein the gas hourly space velocity (GHSV) in the contacting in (iii) is preferably in the range from 1 to 30,000 h-¹, preferably from 500 to 25,000 h-¹, preferably from 1,000 to 20,000 h-¹, more preferably from 1,500 to 10,000 h-¹, more preferably from 2,000 to 5,000 h-¹.
71. The method of any one of embodiments 61 to 70, wherein the one or more olefins and/or one or more hydrocarbons optionally provided in (ii) and/or optionally recycled to (ii) comprise one or more selected from the group consisting of ethylene, (C₄—C,)olefins, (C₄—C,)hydrocarbons, and mixtures of two or more thereof, and preferably from the group consisting of ethylene, (C₄-C₅)olefins, (C₄-C₅)hydrocarbons, and mixtures of two or more thereof.
72. Use of a zeolitic material according to any one of embodiments 1 to 20 and 60 as a molecular sieve, as an adsorbent, for ion-exchange, or as a catalyst and/or as a catalyst support, preferably as a catalyst for the selective catalytic reduction (SCR) of nitrogen oxides NOx; for the oxidation of NH₃, in particular for the oxidation of NH₃ slip in diesel systems; for the decomposition of N₂0; as an additive in fluid catalytic cracking (FCC) processes; and/or as a catalyst in organic conversion reactions, preferably as a hydrocracking catalyst, as an alkylation catalyst, as an isomerization catalyst, or as a catalyst in the conversion of alcohols to olefins, and more preferably in the conversion of oxygenates to olefins.
73. The use of embodiment 72, wherein the zeolitic material is used in a methanol-to-olefin process (MTO process), in a dimethylether to olefin process (DTO process), methanol-togasoline process (MTG process), in a methanol-to-hydrocarbon process, in a methanol to aromatics process, in a biomass to olefins and/or biomass to aromatics process, in a methane to benzene process, for alkylation of aromatics, or in a fluid catalytic cracking process (FCC process), preferably in a methanol-to-olefin process (MTO process) and/or in a dimethylether to olefin process (DTO process), and more preferably in a methanol-topropylene process (MTP process), in a methanol-to-propylene/butylene process (MT3/4 process), in a dimethylether-to-propylene process (DTP process), in a dimethylether-topropylene/butylene process (DT3/4 process), and/or in a dimethylether-toethylene/propylene (DT2/3 process).

The present invention is further illustrated by the following examples and reference examples.

### EXPERIMENTAL SECTION

### Reference Example 1: Determination of Molecular Weight of the Organotemplate

The molecular weight of the bromide salt form of the template was measured with Viscotek TDA305max GPC System equipped with CGuard+1× C-L column set and RI/RALS/IV-DP detectors. Pullulan (Malvern) was used as standard sample. An aqueous solution of acetic acid (5 volume-% of HAc in water) was used as solvent. Inject volume was 100 μL. The temperature of column and detectors were 45° C.

### Reference Example 2: Determination of X-Ray Diffraction Pattern

X-ray powder diffraction (XRD) patterns of a calcined zeolitic material were measured with a Rigaku Ultimate VI X-ray diffractometer (40 kV, 40 mA) using CuKₐ (λ=0.15406 nm (1.5406 Å)) radiation.

### Reference Example 3: Determination of Sample Composition

The sample composition was measured by ICP mass spectrometry with a Perkin-Elmer 3300DV emission spectrometer.

### Reference Example 4: Scanning Electron Microscopy

Scanning electron microscopy (SEM) experiments were performed on Hitachi SU-8010 electron microscopes.

### Reference Example 5: Transmission Electron Microscopy

Transmission electron microscopy experiments were obtained on a JEOL 2100Plus at 200 kV with TVIPS F416 camera. The plane group symmetries of high-resolution TEM images along [001] and [100] zone axes are p2mmand cm, respectively. The high-resolution TEM images were simulated using eMap ([001]: focus −20 nm and thickness 28.6 nm; [100]: focus −120 nm and thickness 2.6 nm).

### Reference Example 6: MAS NMR

²⁷AI, ²⁹Si and ¹⁹F and solid MAS NMR spectra were recorded on a Varian Infinity Plus 400 spectrometer. ¹³C liquid NMR spectrum was recorded on a Bruker Avance 500 spectrometer using a 5 mm QNP probe equipped with z-gradient coil.

### Reference Example 7: Determination of Acidity with NH₃-TPD

The acidity of COE-7 zeolite was measured by temperature-programmed-desorption of NH₃ (NH₃-TPD), which was conducted on a TP-5076 instrument (Xianquan, Tianjin, China) equipped with a TCD detector. Typically, 0.2 g catalyst was loaded into a quartz tube reactor and pretreated at 600° C. for 30 min under He. After being cooled to 120° C., the sample was exposed to NH₃ for 30 min. This was followed by purging with a He flow for 30 min at 120° C. to remove physisorbed NH₃. Then, the sample was heated from 120° C. to 600° C. at a rate of 10° C./min, and the desorbed NH₃ was monitored by the TCD. TG-DTA analysis was finished on SDT Q600 thermal analysis system from room temperature to 800° C. with 10° C./min growth rate under air condition.

### Reference Example 8: Synthesis of Organotemplate OSDA1

As a typical example for the synthesis of the organotemplate, 17.231 g of N,N,N′,N′-tetramethylhexane-1,6-diamine (0.1 mol; C₁₀H2₄N2; AR; 98%; Shanghai Macklin Biochemical Co., Ltd.) were mixed with 21.591 g of 1,4-dibromobutane (0.1 mol; C₄HsBr₂; AR; 98%; Aladdin Chemical Co., Ltd.) and 50 mL of ethanol (C₂H5OH; AR; Sinopharm Chemical Reagent Co., Ltd.). The mixture was heated under reflux for 12 h. Then, the solvent was evaporated, and the white solid precipitate was washed with ether and dried under vacuum. The gel permeation chromatography (GPC) analysis showed that bromide salt form of the template had a molecular weight between 4291 and 8669 (n=11 to 22). The bromide cation was converted to hydroxide form using anion exchange resin (IRN-78; Sigma-Aldrich Co., Ltd.) in deionized water, and the obtained solution was titrated using 0.1 M HCl. The organotemplate used herein as organic structure directing agent is also designated herein as OSDA1.

### Reference Example 9: Synthesis of Seed Crystals

The seed crystals used for the preparation of a zeolitic material having the ITH framework structure type were prepared according to the method as disclosed in Chinese Journal of Chemistry 2017, 35 (5), 572-576. Thus, the seed crystals were all-silica zeolites being free of alumina in their framework structure.

### Example 1: Synthesis of a Zeolitic Material Having the ITH Framework Structure Type (COE-7 Zeolite)

As a typical run for synthesis of COE-7 zeolite, boehmite (1-24 mg; 0.01-0.16 mmol Al₂O₃; Al₂O₃ content of 70 weight-%; Liaoning Hydratight Co., Ltd.) and 10.67 g of organic template solution (OSDA1, 0.36 mol/L OH—) were mixed together. Then, 0.935 g of fumed silica (15.58 mmol; Shanghai Tengmin Industrial Co., Ltd.) was added under stirring condition. After stirring the mixture for another 6 h, 0.05 g of siliceous ITH zeolite seeds prepared in accordance with Reference Example 9, and 0.190 mL of HF (40 weight-%; 3.3 mmol; AR; Aladdin Chemical Co., Ltd.) were added. After evaporating partial water, the final molar ratio of the reaction mixture was 1.0 SiO₂: 0-0.01 Al₂O₃: 0.005 OSDA1:0.2 HF: 1-3 H₂O. Finally, the mixture was transferred into a Teflon-lined autoclave and heated at 160° C. for 10 days under rotation condition (50 rpm). After that, the solid zeolite product was filtered, washed with deionized water, dried at 100° C. and calcined at 550° C. for 5 h. The obtained zeolite was denoted as COE-7-x, where x was the Si/Al ratios in the reaction mixture. After hydrothermal treatment of a H-COE-7 zeolite sample at 800° C. with 10% H₂O for 5 h, the calcined zeolite product was obtained.

### Example 2: Synthesis of a Zeolitic Material Having the ITH Framework Structure Type (COE-7-100)

COE-7-100 was obtained in accordance with the preparation method of Example 1 when using a reaction mixture having a molar ratio of hydrogen fluoride to silica, HF: SiO₂, of 0.25 and a molar ratio of water to silica, H₂O: SiO₂, of 3. COE-7-100 had a molar ratio of silica to alumina, SiO₂: Al₂O₃, of 140. Alternatively, a molar ratio of water to silica, H₂O: SiO₂, of 1 was used leading also to COE-7-100 having a molar ratio of silica to alumina, SiO₂: Al₂O₃, of 140. SEM and TEM measurements according reference examples 4 and 5, respectively, showed that the COE-7-100 zeolite had nanosheet-like morphology with the thickness of about 30 nm. The BET surface area was measured to be about 400 m²/g and the micropore volume to be 0.15 cm³/g.

In addition, the mesopore volume was determined as being 0.22 cm³/g. The ²⁹Si MAS NMR spectrum of the COE-7-100 zeolite revealed four peaks with the chemical shift centered at −116.8, −114.2, −111.3, and −105.4 ppm. The first three peaks are assigned to Si(4Si)species, and the fourth one is attributed to the Si(3Si,OH) and/or Si(3Si,1AI). The ¹⁹F MAS NMR spectrum of the COE-7-100 zeolite displayed two peaks at −35.5 and −63.8 ppm, which are assigned to the fluoride species in the double four member rings and the [4¹526²] cage, respectively. The ²⁷AI MAS NMR spectrum of the COE-7-100 zeolite displayed a peak having a maximum at 53.3 ppm associated with the four-coordinated aluminum species in the ITH zeolite framework, whereby no peak was observed at around 0 ppm. The temperature programmed desorption of ammonia (NH₃-TPD) curve of the H-COE-7-100 zeolite showed two desorption peaks centered at about 185° C. and 390° C.

### Example 3: Synthesis of a Zeolitic Material Having the ITH Framework Structure Type (COE-7-75)

COE-7-75 was obtained in accordance with the preparation method of Example 1 when using a molar ratio of hydrogen fluoride to silica, HF: SiO₂, of 0.25 and a molar ratio of water to silica, H₂O: SiO₂, of 1 in the reaction mixture. COE-7-75 had a molar ratio of silica to alumina, SiO₂: Al₂O₃, of 114.

### Example 4: Synthesis of a Zeolitic Material Having the ITH Framework Structure Type (COE-7-150)

COE-7-150 was obtained in accordance with the preparation method of Example 1 when using a molar ratio of hydrogen fluoride to silica, HF: SiO₂, of 0.25 and a molar ratio of water to silica, H₂O: SiO₂, of 3 in the reaction mixture. COE-7-150 had a molar ratio of silica to alumina, SiO₂: Al₂O₃, of 188.

### Example 5: Synthesis of a Zeolitic Material Having the ITH Framework Structure Type (COE-7-200)

COE-7-200 was obtained in accordance with the preparation method of Example 1 when using a molar ratio of hydrogen fluoride to silica, HF: SiO₂, of 0.25 and a molar ratio of water to silica, H₂O: SiO₂, of 3 in the reaction mixture. COE-7-200 had a molar ratio of silica to alumina, SiO₂: Al₂O₃, of 240.

### Example 6: Synthesis of a Zeolitic Material Having the ITH Framework Structure Type (COE-7-Si)

COE-7-Si was obtained in accordance with the preparation method of Example 1 when using a molar ratio of hydrogen fluoride to silica, HF: SiO₂, of 0.25 and a molar ratio of water to silica, H₂O: SiO₂, of 3 in the reaction mixture. A molar ratio of silica to alumina, SiO₂: Al₂O₃, of COE-7-Si was not determined.

### Example 7: Synthesis of a Zeolitic Material Having the ITH Framework Structure Type

For synthesis of an Al₂O₃-containing zeolitic material having framework structure type ITH with a SiO₂/Al₂O₃ molar ratio of 122 (COE-7-50) using a zeolitic material having framework structure type LTA as source for X₂03, 0.043 g zeolite LTA (Si/Al=1) and 10.67 g of an organic template solution (OSDA1, 0.36 mol/L OH—) were mixed. Then, 0.912 g of fumed silica (Shanghai Tengmin Industrial Co., Ltd.) was added under stirring. After stirring the mixture for another 6 h, 0.05 g of siliceous ITH zeolite seeds prepared in accordance with Reference Example 9, and 0.190 mL of HF (40 weight-% in water, 3.3 mmol; AR; Aladdin Chemical Co., Ltd.) were added.

After partial water evaporation from starting gel (H₂O:SiO₂>30), the final molar ratios of the mixture were 1.0 SiO₂: 0.01 Al₂O₃: 0.005 OSDA1: 0.2 HF: 1 H₂O. Finally, the mixture was transferred into a Teflon-lined autoclave and heated at 175° C. for 7 days under rotation condition (50 rpm). After that, the solid zeolite product was filtered, washed with deionized water, dried at 100° C. and calcined at 550° C. for 5 h. The BET surface area of the resulting zeolite was measured to be about 320 m²/g and the micropore volume to be 0.13 cm³/g. In addition, the mesopore volume was determined as being 0.29 cm³/g. The resulting zeolitic material was also characterized via X-ray diffraction analysis according to Reference Example 2, the powder X-ray diffraction pattern is shown in FIG. 2.

### Example 8: Synthesis of a Zeolitic Material Having the ITH Framework Structure Type

For synthesis of an Al₂O₃-containing zeolitic material having framework structure type ITH with a SiO₂/AI₂O₃ molar ratio of 64 (COE-7-20) using a zeolitic material having framework structure type LTA as source for X₂03, 0.086 g zeolite LTA (Si/Al=1) and 10.67 g of organic template solution (OSDA1, 0.36 mol/L OH—) were mixed. Then, 0.888 g of fumed silica (Shanghai Tengmin Industrial Co., Ltd.) was added under stirring. After stirring the mixture for another 6 h, 0.05 g of siliceous ITH zeolite seeds prepared in accordance with Reference Example 9, and 0.190 mL of HF (40 weight-% in water, 3.3 mmol; AR; Aladdin Chemical Co., Ltd.) were added. After partial water evaporation from starting gel (H₂O:SiO₂>30), the final molar ratios of the mixture were 1.0 SiO₂: 0.025 Al₂O₃: 0.005 OSDA1: 0.2 HF: 1 H₂O. Finally, the mixture was transferred into a Teflon-lined autoclave and heated at 175° C. for 7 days under rotation condition (50 rpm). After that, the solid zeolite product was filtered, washed with deionized water, dried at 100° C. and calcined at 550° C. for 5 h. The BET surface area of the resulting zeolite was measured to be about 324 m²/g and the micropore volume to be 0.14 cm³/g. In addition, the mesopore volume was determined as being 0.29 cm³/g.

### Example 9: Synthesis of a Zeolitic Material Having the ITH Framework Structure Type

For synthesis of an Al₂O₃-containing zeolitic material having framework structure type ITH with a SiO₂/Al₂O₃ molar ratio of 136 (COE-7-50) using a zeolitic material having framework structure type LTA as source for X₂03, 0.043 g zeolite LTA (Si/Al=1) and 10.67 g of organic template solution (OSDA1, 0.36 mol/L OH—) were mixed. Then, 0.912 g of fumed silica (Shanghai Tengmin Industrial Co., Ltd.) was added under stirring. After stirring the mixture for another 6 h, 0.05 g of siliceous ITH zeolite seeds prepared in accordance with Reference Example 9, and 0.190 mL of HF (40 weight-% in water, 3.3 mmol; AR; Aladdin Chemical Co., Ltd.) were added. After partial water evaporation from starting gel (H₂O:SiO₂>30), the final molar ratios of the mixture were 1.0 SiO₂: 0.01 Al₂O₃: 0.005 OSDA1: 0.2 HF: 1 H₂O. Finally, the mixture was transferred into a Teflon-lined autoclave and heated at 175° C. for 7 days under rotation condition (50 rpm). After that, the solid zeolite product was filtered, washed with deionized water, dried at 100° C. and calcined at 550° C. for 5 h. The BET surface area of the resulting zeolite was measured to be about 304 m²/g and the micropore volume to be 0.14 cm³/g. In addition, the mesopore volume was determined as being 0.29 cm³/g.

### Comparative Example 1: Synthesis of AI,Ge-ITH Zeolite

A conventional AI—Ge-ITH zeolite was synthesized under hydrothermal conditions. In a typical run for synthesizing conventional AI—Ge-ITH zeolite, 0.078 g of GeO₂ (99.999%; metal basis; 200 mesh; Aladdin Chemical Co., Ltd.) was dissolved in 1.946 g of aqueous hexamethonium hydroxide solution (HM(OH)₂; 25 weight-%; Kente Catalysis Co., Ltd.). Then, 0.034 g of aluminum isopropoxide (Al₂O₃ of 24.7 weight-%; Sinopharm Chemical Reagent Co., Ltd.) was added to the above solution. After stirring for 1 h, 3.105 g of tetraethyl orthosilicate (TEOS, 99%, Aladdin Chemical Co., Ltd.) were added. After that, the mixture was stirred overnight. Finally, 0.190 mL of an aqueous hydrofluoric acid solution (HF; AR; 40 weight-%; Aladdin Chemical Co., Ltd.) was added into the mixture, then the gel was weighted and placed at room temperature to evaporate 3.929 g of water. The final gel was transferred into a Teflon-lined autoclave and placed at 150° C. for 7 days under rotation conditions (50 rpm). The solid zeolite product was filtered, washed with deionized water, dried at 100° C., and calcined at 550° C. for 5 h. After hydrothermal treatment of H-AI—Ge-ITH zeolite at 800° C. with 10% H₂O for 5 h, the calcined zeolite product was obtained.

### Comparative Example 2: Synthesis of ZSM-5 Zeolite

A conventional ZSM-5 zeolite was synthesized according to the method disclosed by C. Zhang et al. “An Efficient, Rapid, and Non-Centrifugation Synthesis of Nanosized Zeolites by Accelerating the Nucleation Rate” in J. Mater. Chem. A 2018, 6(42), 21156-21161.

### Example 10: Catalytic Testing—Methanol-to-Olefin Reaction

A methanol-to-olefin (MTO) reaction was carried out at 480° C. and 1 atmospheric pressure (101.325 Pa) in a fixed-bed microreactor. A zeolite sample (500 mg, 20-40 mesh) was pretreated in flowing nitrogen at 500° C. for 2 h and then cooled down to reaction temperature. The methanol was continuously injected into the catalyst bed by a pump with a weight hourly space velocity (WHSV) of 1 hW. The products were analyzed by online gas chromatography (Agilent 6890N) with FID detector using PLOT-AI₂O₃ column.

The results of the catalytic testing are shown in table 1 below. As can be gathered from table 1a zeolitic material according to the present invention in particular displays a comparatively higher selectivity towards propylene as well as towards butylene compared to a conventional ZSM-5 zeolite while showing similar total conversion.

TABLE 1
Results of MTO reactions for a reaction time
of 2 hours at 480° C.

| | | Conversion | Selectivity [%] | | |
|---|---|---|---|---|---|
| Sample | Si/Al | [%] | C2= | C3= | C4= |
| COE-7 | 70 | 100 | 13.3 | 46.7 | 16.6 |
| ZSM-5 | 90 | 100 | 23.1 | 33.2 | 11.6 |

Further, as can be gathered from FIG. 1, a zeolitic material according to the present invention (COE-7-100 zeolite) displays higher selectivity for propene and longer catalytic lifetime than a conventional ZSM-5 zeolite, showing its potential importance with respect to selective production of propylene in the industrial applications.

### BRIEF DESCRIPTION OF FIGURES

FIG. 1: shows the catalytic performance of a zeolitic material according to the present invention relative to a conventional ZSM-5 zeolite in MTO reaction. In particular, dependences of methanol conversion and product selectivity on reaction time in MTO over the COE-7 zeolite (solid labels) and ZSM-5 (hollow labels) at 480 0° C. are shown.

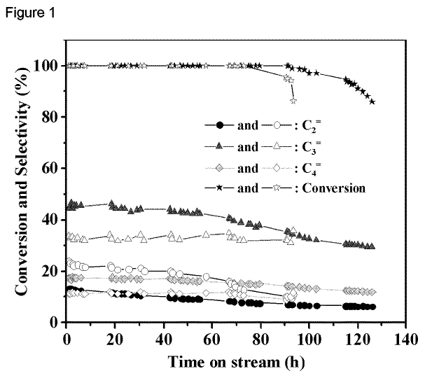

FIG. 2: shows the powder X-ray diffraction pattern of a zeolitic material according to Example 7. On the abscissa, the 2theta angle is shown in degree and on the ordinate the intensity is shown in arbitrary units.

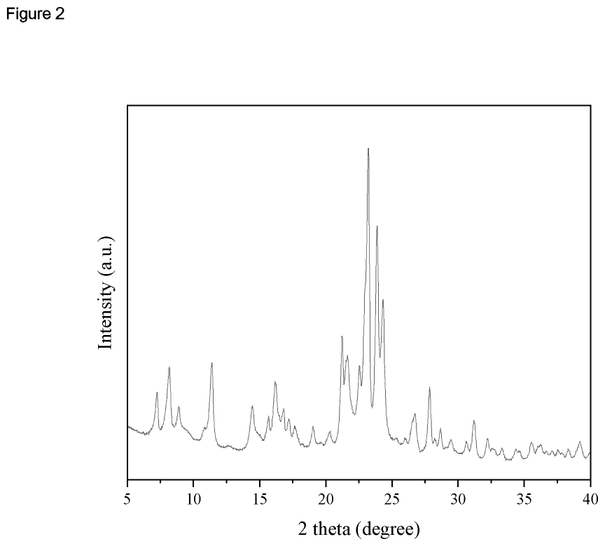

### CITED LITERATURE

CN 106698456 A
C. Zhang et al. “An Efficient, Rapid, and Non-Centrifugation Synthesis of Nanosized Zeolites by Accelerating the Nucleation Rate” in J. Mater. Chem. A 2018, 6 (42), 21156-21161
P. Zeng et al. “On the synthesis and catalytic cracking properties of AI-ITQ-13 zeolites” in Microporous and Mesoporous Materials 2017, 246, 186
G. Xu et al. “Synthesis of pure silica ITQ-13 zeolite using fumed silica as silica source” in Microporous and Mesoporous Materials 2010, 129, 278
X. Liu et al. “Synthesis of all-silica zeolites from highly concentrated gels containing hexamethonium cations” in Microporous and Mesoporous Materials 2012, 156, 257
R. CastaAeda et al. “Direct synthesis of a 9×10 member ring zeolite (AI-ITQ-13): A highly shape-selective catalyst for catalytic cracking” in Journal of Catalysis 2006, 238, 79-87
L. Liu et al. “Oriented control of Al locations in the framework of AI—Ge-ITQ-13 for catalyzing methanol conversion to propene” in Journal of Catalysis 2016, 344, 242-251
H. Ma, “Reaction mechanism for the conversion of methanol to olefins over H-ITQ-13 zeolite: a density functional theory study” in Catalysis Science and Technology 2018, 8, 521
A. Corma et al. “A Zeolite Structure (ITQ-13) with Three Sets of Medium-Pore Crossing Channels Formed by 9- and 10-Rings” in Angew. Chem./nt. Ed. 2003, 42 (10), 1156-1159
Q. Wu et al. “Solvent-Free Synthesis of ITQ-12, ITQ-13, and ITQ-17 zeolites” in Chin. J.

Chem. 2017, 35, 572

1. A zeolitic material having the ITH framework structure type,
wherein the zeolitic material comprises YO₂ and optionally X₂O₃ in its framework structure,
wherein Y is a tetravalent element and X is a trivalent element,
wherein the framework structure of the zeolitic material comprises less than 4 weight-% of Ge calculated as GeO₂ and based on 100 weight-% of YO₂ contained in the framework structure,
wherein the zeolitic material comprises less than 1.5 weight-% of B calculated as B₂03 and based on 100 weight-% of X₂O₃ contained in the framework structure, and wherein the zeolitic material has a molar ratio YO₂: X₂O₃ of equal or greater than 50.

2. The zeolitic material of claim 1, wherein the zeolitic material comprises YO₂ and X₂O₃ in its framework structure, wherein the zeolitic material has a molar ratio YO₂: X₂03 in the range of from 100 to 250.

3. The zeolitic material of claim 1, wherein Y comprises Si,
wherein the ²⁹Si MAS NMR of the zeolitic material comprises:
a first peak having a maximum in the range of from −101.0 to −107.0 ppm;
a second peak having a maximum in the range of from −105.0 to −112.7 ppm;
a third peak having a maximum in the range of from −111.0 to −116.0 ppm; and
a fourth peak having a maximum in the range of from −115.1 to −118.4 ppm.

4. The zeolitic material of claim 1, wherein
the zeolitic material displays an X-ray powder diffraction pattern comprising at least the following reflections:

| | Diffraction angle 2θ/° |
|---|---|
| Intensity (%) | [Cu K(alpha 1)] |
| 100 | [7.00-7.20] |
| [33-53] | [7.82-8.02] |
| [22-42] | [8.60-8.80] |
| [42-62] | [11.12-11.32] |
| [28-48] | [15.38-15.58] |
| [19-39] | [16.05-16.25] |
| [19-39] | [16.48-16.68] |
| [17-37] | [20.94-21.14] |
| [14-34] | [21.40-21.60] |
| [77-97] | [22.92-23.12] |
| [57-77] | [23.54-23.74] |
| [31-51] | [23.90-24.10] |

wherein 100% relates to the intensity of the maximum peak in the X-ray powder diffraction pattern.

5. A process for the preparation of a zeolitic material having the ITH framework structure type, wherein the process comprises
(1) preparing a mixture comprising one or more organotemplates as structure directing agents, one or more sources of YO₂, optionally one or more sources of X₂O₃, seed crystals, and a solvent system, wherein Y is tetravalent element and X is a trivalent element;
(2) heating the mixture obtained in (1) for crystallizing a zeolitic material having the ITH framework structure type comprising YO₂ and optionally X₂O₃ in its framework structure;
wherein the one or more organotemplates comprise a polymeric cation comprising a unit of formula (I):
[R¹R²N⁺−R⁵−N⁺R³R⁴−R⁶]ₙ (I);
wherein R¹, R², R³, and R⁴ independently from one another is (C1-C₄)alkyl;
wherein R⁵ is selected from the group consisting of tetramethylene, pentamethylene, hexamethylene, and heptamethylene;
wherein R⁶ is selected from the group consisting of trimethylene, tetramethylene, and pentamethylene;
wherein n is a natural number in the range of from 1 to 50.

6. The process of claim 5, wherein the organotemplate: YO₂ molar ratio of the one or more organotemplates to the one or more sources of YO₂ calculated as YO₂ in the mixture prepared in (1) and heated in (2) is in the range of from 0.001 to 0.5.

7. The process of claim 5, wherein the seed crystals comprise one or more zeolitic materials having the ITH framework structure type.

8. The process of claim 5, wherein the amount of seed crystals comprised in the mixture prepared in (1) is in the range of from 0.1 to 15 weight-% based on 100 weight-% of the one or more sources of YO₂ calculated as YO₂.

9. The process of claim 5, wherein the mixture comprises one or more sources for X₂03, wherein the X₂O₃: YO₂ molar ratio of the one or more sources of X₂O₃ calculated as X₂O₃ to the one or more sources of YO₂ calculated as YO₂ in the mixture prepared in (1) and heated in (2) is in the range of from 0.001 to 0.1.

10. The process of claim 5, wherein the mixture prepared in (1) further comprises one or more sources of fluoride, wherein the fluoride: YO₂ molar ratio of the one or more sources of fluoride calculated as the element to the one or more sources of YO₂ calculated as YO₂ in the mixture prepared in (1) and heated in (2) is in the range of from 0.01 to 2.

11. The process of claim 5, wherein the one or more organotemplates are prepared according to a process comprising
(a) preparing a reaction mixture comprising a compound having the formula (II)
R¹R²N⁺⁻R⁵−N+R³R⁴ (II)
a compound having the formula (III)
Rₐ−R⁶−R_{b} (III)
and a solvent system, to obtain a reaction mixture;
(b) heating the reaction mixture, to obtain a mixture comprising one or more organotemplates;
wherein R¹, R², R³, and R⁴ independently from one another is (C₁-C₄)alkyl;
wherein R^{S} is selected from the group consisting of tetramethylene, pentamethylene, hexamethylene, and heptamethylene;
wherein R⁶ is selected from the group consisting of trimethylene, tetramethylene, and pentamethylene; and
wherein Rₐ and R_{b} independently from each other is selected from the group consisting of F, Cl, Br, I, tosyl (OTs), mesyl, triflourmethansulfonate (OTf), and OH.

12. The process of claim 5, wherein the process further comprises
(3) isolating the zeolitic material obtained in (2),
and/or
(4) washing the zeolitic material obtained in (2) or (3),
and/or
(5) drying the zeolitic material obtained in (2), (3), or (4), in a gas atmosphere,
and/or
(6) calcining the zeolitic material obtained in (2), (3), (4) or (5) in a gas atmosphere,
and/or
(7) subjecting the zeolitic material obtained in (2), (3), (4), (5) or (6) to an ionexchange procedure with one or more metal cations M,
wherein the steps (3) and/or (4) and/or (5) and/or (6) and/or (7) can be conducted in any order.

13. A zeolitic material having the ITH framework structure type obtainable and/or obtained from the process of claim 5.

14. A method for the conversion of oxygenates to olefins comprising
(i) providing a catalyst according to claim 1;
(ii) providing a gas stream comprising one or more oxygenates and optionally one or more olefins and/or optionally one or more hydrocarbons;
(iii) contacting the catalyst provided in (i) with the gas stream provided in (ii) and converting one or more oxygenates to one or more olefins and optionally to one or more hydrocarbons;
(iv) optionally recycling one or more of the one or more olefins and/or of the one or more hydrocarbons contained in the gas stream obtained in (iii) to (ii).

15. (canceled)

16. A molecular sieve comprising a zeolitic material according to claim 1.

17. An adsorbent comprising a zeolitic material according to claim 1.

18. A catalyst comprising a zeolitic material according to claim 1.

19. A catalyst support comprising a zeolitic material according to claim 1.
